SEQUENCE LISTING (37 sequences)

SEQ ID NO: 1 - protein, 335 aa, Homo sapien
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 2 - DNA, 1789 bp, Homo sapien
ggttcaggaacctgctggttctgatacataaatcagacagcctctgctgcatgacacgaagcttgcttctgcctggcatctgtgagcagctgccaggctccggccaggatcccttccttctcctcattggctgatggatcccaaggggctcctctccttgaccttcgtgctgtttctctccctggcttttggggcaagctacggaacaggtgggcgcatgatgaactgcccaaagattctccggcagttgggaagcaaagtgctgctgcccctgacatatgaaaggataaataagagcatgaacaaaagcatccacattgtcgtcacaatggcaaaatcactggagaacagtgtcgagaacaaaatagtgtctcttgatccatccgaagcaggccctccacgttatctaggagatcgctacaagttttatctggagaatctcaccctggggatacgggaaagcaggaaggaggatgagggatggtaccttatgaccctggagaaaaatgtttcagttcagcgcttttgcctgcagttgaggctttatgagcaggtctccactccagaaattaaagttttaaacaagacccaggagaacgggacctgcaccttgatactgggctgcacagtggagaagggggaccatgtggcttacagctggagtgaaaaggcgggcacccacccactgaacccagccaacagctcccacctcctgtccctcaccctcggcccccagcatgctgacaatatctacatctgcaccgtgagcaaccctatcagcaacaattcccagaccttcagcccgtggcccggatgcaggacagacccctcagaaacaaaaccatgggcagtgtatgctgggctgttagggggtgtcatcatgattctcatcatggtggtaatactacagttgagaagaagaggtaaaacgaaccattaccagacaacagtggaaaaaaaaagccttacgatctatgcccaagtccagaaaccaggtcctcttcagaagaaacttgactccttcccagctcaggacccttgcaccaccatatatgttgctgccacagagcctgtcccagagtctgtccaggaaacaaattccatcacagtctatgctagtgtgacacttccagagagctgacaccagagaccaacaaagggactttctgaaggaaaatggaaaaaccaaaatgaacactgaacttggccacaggcccaagtttcctctggcagacatgctgcacgtctgtacccttctcagatcaactccctggtgatgtttcttccacatacatctgtgaaatgaacaaggaagtgaggcttcccaagaatttagcttgctgtgcagtggctgcaggcgcagaacagagcgttacttgataacagcgttccatctttgtgttgtagcagatgaaatggacagtaatgtgagttcagactttgggcatcttgctcttggctggaactgataataaaaatcagactgaaagccaggacatctgagtacctatctcacacactgaccaccagtcacaaagtctggaaaagtttacattttggctatctttactttgttctgggagctgatcatgataacctgcagacctgatcaagcctctgtgcctcagtttctctctcaggataaagagtgaatagaggccgaagggtgaatttcttattatacataaaacactctgatattattgtataaaggaagctaagaatattattttatttgcaaaacccagaagctaaaaagtcaataaacagaaagaatgattttgagaaa

SEQ ID NO: 3 - protein, 329 aa, Mus musculus
MDPKGSLSWRILLFLSLAFELSYGTGGGVMDCPVILQKLGQDTWLPLTNEHQINKSVNKSVRILVTMATSPGSKSNKKIVSFDLSKGSYPDHLEDGYHFQSKNLSLKILGNRRESEGWYLVSVEENVSVQQFCKQLKLYEQVSPPEIKVLNKTQENENGTCSLLLACTVKKGDHVTYSWSDEAGTHLLSRANRSHLLHITLSNQHQDSIYNCTASNPVSSISRTFNLSSQACKQESSSESSPWMQYTLVPLGVVIIFILVFTAIIMMKRQGKSNHCQPPVEEKSLTIYAQVQKSGVRSMPHLAGVSVIFRTGFLIAALHTTMVLQGLLE

SEQ ID NO: 4 - DNA, 1288 bp, Mus musculus
tcctgccgagctgagctgagctgagctcacagctgggaccctgtctgcgattgctggctaatggatcccaaaggatccctttcctggagaatacttctgtttctctccctggcttttgagttgagctacggaacaggtggaggtgtgatggattgcccagtgattctccagaagctgggacaggacacgtggctgcccctgacgaatgaacatcagataaataagagcgtgaacaaaagtgtccgcatcctcgtcaccatggcgacgtccccaggaagcaaatccaacaagaaaattgtgtcttttgatctctctaaagggagctatccagatcacctggaggatggctaccactttcaatcgaaaaacctgagcctgaagatcctcgggaacaggcgggagagtgaaggatggtacttggtgagcgtggaggagaacgtttctgttcagcaattctgcaagcagctgaagctttatgaacaggtctcccctccagagattaaagtgctaaacaaaacccaggagaacgagaatgggacctgcagcttgctgttggcctgcacagtgaagaaaggggaccatgtgacttacagctggagtgatgaggcaggcacccacctgctgagccgagccaaccgctcccacctcctgcacatcactcttagcaaccagcatcaagacagcatctacaactgcaccgcaagcaaccctgtcagcagtatctctaggaccttcaacctatcatcgcaagcatgcaagcaggaatcctcctcagaatcgagtccatggatgcaatatactcttgtaccactgggggtcgttataatcttcatcctggttttcacggcaataataatgatgaaaagacaaggtaaatcaaatcactgccagccaccagtggaagaaaaaagccttactatttatgcccaagtacagaaatcaggggtacgttctatgcctcaccttgcgggagtgtctgtcatatttcgcacaggatttctgatagctgccttgcacacaaccatggtcctgcagggactcctagagtagatgaacttaagaaagcagaaaagtcaagaacaagagctcccccagtgtcactgacccttatattgtttgaacttgtagaaaacagtgacaatccagttgcctgcttttttgtagtaatgagatgaagttgtaagaacatgaccttgatactttccagcagtggtattctagattgagaactcctagaaattctcaacttccttcttggagtgtgtaggcaagggaaggaggtgaagac

SEQ ID NO: 5 - protein, 365 aa, Homo sapien
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 6 - DNA, 2308 bp, Homo sapien
atgctggggcaagtggtcaccctcatactcctcctgctcctcaaggtgtatcagggcaaaggatgccagggatcagctgaccatgtggttagcatctcgggagtgcctcttcagttacaaccaaacagcatacagacgaaggttgacagcattgcatggaagaagttgctgccctcacaaaatggatttcatcacatattgaagtgggagaatggctctttgccttccaatacttccaatgatagattcagttttatagtcaagaacttgagtcttctcatcaaggcagctcagcagcaggacagtggcctctactgcctggaggtcaccagtatatctggaaaagttcagacagccacgttccaggtttttgtatttgataaagttgagaaaccccgcctacaggggcaggggaagatcctggacagagggagatgccaagtggctctgtcttgcttggtctccagggatggcaatgtgtcctatgcttggtacagagggagcaagctgatccagacagcagggaacctcacctacctggacgaggaggttgacattaatggcactcacacatatacctgcaatgtcagcaatcctgttagctgggaaagccacaccctgaatctcactcaggactgtcagaatgcccatcaggaattcagattttggccgtttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcctagttgctgcagcaattctcacctttcttgcacatcagcatctgctttgggaattggcacagtggatgacggcacaggagtctctatagaacagttcctagtctggagaggatatggaaatttgttcttgttctatattttgttttgaaaatgatgtctaacaaccatgataagagcaaggctgttaaataatatcttccaatttacagatcagacatgaatgggtggaggggttaggttgttcacaaaaggccacattccaagtatttgtaatctagaaagtgttatgtaagtgatgttattagcatcgagattccctccacctgattttcaagctgtcacttgtttccttttctcccctctctgggttgactgcatttctagactctcgccggcccaggcccatcttccaaagcaagaggaaggaatgataatggtgactcaggggaagaagaaacagccctcctctgaaagcctggactgtccggctgtgaactggctggcaggttctgcacgtgggtgggggccagggcctgggctttactcaattgcagagaaaaaactttctccctgcatctcacacctttacctctgcccagttggccaccagggggagtgggctgaagggagagtagatggtgcaaagcaagcccatctctgagtagaaaaatcacccagagcacatgctgacctgataactggggtgttgagaccagctttgtccatggtatgatgtttgatttatgaagacgcattgttagaaatccatttggcttcttcatagaagtggcttcccagaggaagaggcctctcagaaaccatgttctatttaagttctgagtcctgatgagtgttccccaggatgcacattgaagggagggctcaggcagctgagggctgagaatgaggcagttggaatctagacactatgctgggttccctgagtcgtcaggccagacatttcaacaaggctgtggggagcagggctgtgactctggctgagcccaggaaagcgacaagggtgaactgggagaggacttactcagagaccccaacaggtgatactgcacaaagcctggttcttcaattttcctaccctgtatctaacataggagtttcatataaaacggtgatatcatgcagatgcagtctgaattccttgcctgaattaaatttatgatcctctccaaaaaaaaaaaaaaagggcggccgc

SEQ ID NO: 7 - protein, 340 aa, Mus musculus
MLGQAVLFTTFLLLRAHQGQDCPDSSEEVVGVSGKPVQLRPSNIQTKDVSVQWKKTEQGSHRKIEILNWYNDGPSWSNVSFSDIYGFDYGDFALSIKSAKLQDSGHYLLEITNTGGKVCNKNFQLLILDHVETPNLKAQWKPWTNGTCQLFLSCLVTKDDNVSYALYRGSTLISNQRNSTHWENQIDASSLHTYTCNVSNRASWANHTLNFTHGCQSVPSNFRFLPFGVIIVILVTLFLGAIICFCVWTKKRKQLQFSPKEPLTIYEYVKDSRASRDQQGCSRASGSPSAVQEDGRGQRELDRRVSEVMFSSLLAFLLHQFPGSTQRGKEKRERAEKNGK

SEQ ID NO: 8 - DNA, 1327 bp, Mus musculus
ggaaattgtggctggacaggcagtgaggtagttctgctgtgtcctgctgacagttagctgcgagcctattgacatctgagcacctggagaagacagggcccccgtagtctgctctgtcctgtggtgatgttggggcaagctgtcctgttcacaaccttcctgctcctcagggctcatcagggccaagactgcccagattcttctgaagaagtggttggtgtctcaggaaagcctgtccagctgaggccttccaacatacagacaaaagatgtttctgttcaatggaagaagacagaacagggctcacacagaaaaattgagatcctgaattggtataatgatggtcccagttggtcaaatgtatcttttagtgatatctatggttttgattatggggattttgctcttagtatcaagtcagctaagctgcaagacagtggtcactacctgctggagatcaccaacacaggcggaaaagtgtgcaataagaacttccagcttcttatacttgatcatgttgagacccctaacctgaaggcccagtggaagccctggactaatgggacttgtcaactgtttttgtcctgcttggtgaccaaggatgacaatgtgagctacgctttgtacagagggagcactctgatctccaatcaaaggaatagtacccactgggagaaccagattgacgccagcagcctgcacacatacacctgcaacgttagcaacagagccagctgggcaaaccacaccctgaacttcacccatggctgtcaaagtgtcccttcgaatttcagatttctgccctttggggtgatcatcgtgattctagttacattatttctcggggccatcatttgtttctgtgtgtggactaagaagaggaagcagttacagttcagccctaaggaacctttgacaatatatgaatatgtcaaggactcacgagccagcagggatcaacaaggatgctctagggcctctggatctccctcggctgtccaggaagatgggaggggacaaagagaattggacaggcgtgtttctgaggtgatgttcagctcccttctagcttttcttctccaccagtttcctggctctacccaaagaggaaaggagaaaagagagagagcagagaaaaatgggaaataggcagaaggggtcacatggtgctgtactagatctgccagggaggaactcagtagaaatccatagcaaagggcagagtgtgtctaccactgccttcccttatagaaaccgttcagggccagctttcctttgtgcattatccacagctggttctaccctattatttaagaactttttcaaa

SEQ ID NO: 9 - protein, 629 aa, Mus musculus
MSQQQIFSPILWIPLLFLLMGLGASGKETPPTVISGMLGGSVTFSLNISKDAEIEHIIWNCPPKALALVFYKKDITILDKGYNGRLKVSEDGYSLYMSNLTKSDSGSYHAQINQKNVILTTNKEFTLHIYEKLQKPQIIVESVTPSDTDSCTFTLICTVKGTKDSVQYSWTREDTHLNTYDGSHTLRVSQSVCDPDLPYTCKAWNPVSQNSSQPVRIWQFCTGASRRKTAAGKTVVGILGEPVTLPLEFRATRATKNVVWVLNTSVISQERRGAATADSRRKPKGSEERRVRTSDQDQSLKISQLKMEDAGPYHAYVCSEASRDPSVRHFTLLVYKRLEKPSVTKSPVHMMNGICEVVLTCSVDGGGNNVTYTWMPLQNKAVMSQGKSHLNVSWESGEHLPNFTCTAHNPVSNSSSQFSSGTICSGPERNKRFWLLLLLVLLLLMLIGGYFILRKKKQCSSLATRYRQAEVPAEIPEPPTGHGQFSVLSQRYEKLDMSAKTTRHQPTPTSDTSSESSATTEEDDEKTRMHSTANSRNQLYDLVTHQDIAHALAYEGQVEYEAITPYDKVDGSMDEEDMAYIQVSLNVQGETPLPQKKEDSNTIYCSVQKPKKTAQTPQQDAESPESPYL

SEQ ID NO: 10 - DNA, 2345 bp, Mus musculus
ctgaaaatccaagaatgagtcagcagcagatattttctcccattctttggattcctctcctcttcctactcatggggctcggagcctctggaaaggaaacacctccaacagtgatatcagggatgctagggggttctgtgactttctccctaaacatctcaaaggatgcagagattgagcatatcatctggaattgtcccccaaaggctcttgctttagtattctacaaaaaagatataactattctggacaaaggatataatggcagactcaaagtcagcgaagatggctactccttgtacatgagcaacctaaccaaaagtgattcaggatcctaccatgctcagataaaccaaaagaatgttatcctcaccacaaataaagagttcacactgcacatctatgagaagctccagaagcctcagatcatcgtggaatctgtgaccccatccgatactgattcctgcaccttcaccctaatctgcactgtaaaggggacaaaggacagtgtccagtacagctggacccgagaggacacccatttaaatacatacgatggaagccacaccctcagggtttcccagagtgtctgtgaccccgacctaccctatacctgcaaagcctggaatccagtcagccaaaacagctcccaacctgtccgcatctggcaattctgcacaggagcctccagaagaaaaacagcagcggggaagactgtggtaggaatcctgggagagccagtgaccctgcccttggaatttcgggccactcgggccacaaagaatgttgtctgggtgttgaacacgtcggttatcagccaagaacggagaggagcagcaacagcggattctcgccgtaagcccaaaggttctgaagaacggagggtgaggacctctgaccaggaccaatccctgaagatcagccagctgaagatggaggacgcgggcccctaccatgcctatgtgtgctcagaggcctcccgagaccccagtgtgagacatttcaccttgcttgtctacaagagactggagaagcccagtgtcaccaagagtcctgtgcacatgatgaacggaatctgcgaggttgtcctgacctgttcagtggacggtggtggaaacaatgtgacatacacatggatgcctctacaaaacaaagctgtcatgtcccaagggaagtcgcacctcaacgtctcctgggaaagtggtgaacacctgcccaacttcacatgcacagcccataaccctgtcagcaacagctccagccagttttcttctgggaccatctgttcaggccctgagagaaacaagaggttttggctcctgctcctcctggttttgctcttgttgatgctcattggcggttacttcattttgaggaaaaagaagcagtgttcgtctttggccaccaggtacagacaagcggaggtcccagctgaaataccagaacccccaactggccatggacaattttctgtgctctcccaacggtatgagaaactagacatgtctgctaagaccaccaggcatcagcctacacccacctcagataccagctctgagagcagcgcaacaacagaagaggatgacgaaaagaccagaatgcacagcactgctaatagtagaaatcagctgtatgacttggtcacccatcaggacattgcacatgccttggcctatgaggggcaagtagaatatgaagcaatcactccatatgataaagtggatgggtctatggatgaagaggacatggcatatatacaagtgtccctgaatgtgcagggagagaccccacttcctcagaagaaagaagactcaaatacaatctactgctctgtgcagaagcctaaaaagacggcacaaacaccacagcaagatgctgagtctcctgaaagcccctacctatgaaaatttcacctgaaaggaaaagcggctgctacttccggggtgaggatttggagaaaaacagacatcataaatcctcctgtttctgcagaaagaacacatcagaattccctgccatggcttagactagcctgaatttgatgccttcctcttcttgtcatcctccttaccccatgagcccattcacaggtcaggcagagtctccttggagcctacaggctttgaacaccctgcctttggatctctttttcttatctggacggccttgtctgcctcagtgggggaggatgcacttagtcctgttgggactggatgtgccagggtgggttggtaccgtctctgaggagaaggggagatgggaattgggggagggatgtgagggagggatgggaggcctgtggtcaatatgtaaagtgaataaataaactaataaagaaaaaaatt

SEQ ID NO: 11 - protein, 610 aa, Homo sapien
LQTSLLFLLMGLRASGKDSAPTVVSGILGGSVTLPLNISVDTEIENVIWIGPKNALAFARPKENVTIMVKSYLGRLDITKWSYSLCISNLTLNDAGSYKAQINQRNFEVTTEEEFTLFVYEQLQEPQVTMKSVKVSENFSCNITLMCSVKGAEKSVLYSWTPREPHASESNGGSILTVSRTPCDPDLPYICTAQNPVSQRSSLPVHVGQFCTDPGASRGGTTGETVVGVLGEPVTLPLALPACRDTEKVVWLFNTSIISKEREEAATADPLIKSRDPYKNRVWVSSQDCSLKISQLKIEDAGPYHAYVCSEASSVTSMTHVTLLIYRRLRKPKITWSLRHSEDGICRISLTCSVEDGGNTVMYTWTPLQKEAVVSQGESHLNVSWRSSENHPNLTCTASNPVSRSSHQFLSENICSGPERNTKLWIGLFLMVCLLCVGIFSWCIWKRKGRCSVPAFCSSQAEAPADTPGYEKLDTPLRPARQQPTPTSDSSSDSNLTTEEDEDRPEVHKPISGRYEVFDQVTQEGAGHDPAPEGQADYDPVTPYVTEVESVVGENTVYAQVFNLQGKTPVSQKEESSATIYCSIRKPQVVPPPQQNDLEIPESPTYENFT

SEQ ID NO: 12 - DNA, 2286 bp, Homo sapien
ctacagacctctctcctcttcctgctcatgggactaagagcctctggaaaggactcagccccaacagtggtgtcagggatcctagggggttccgtgactctccccctaaacatctcagtagacacagagattgagaacgtcatctggattggtcccaaaaatgctcttgctttcgcacgtcccaaagaaaatgtaaccattatggtcaaaagctacctgggccgactagacatcaccaagtggagttactccctgtgcatcagcaatctgactctgaatgatgcaggatcctacaaagcccagataaaccaaaggaattttgaagtcaccactgaggaggaattcaccctgttcgtctatgagcagctgcaggagccccaagtcaccatgaagtctgtgaaggtgtctgagaacttctcctgtaacatcactctaatgtgctccgtgaagggggcagagaaaagtgttctgtacagctggaccccaagggaaccccatgcttctgagtccaatggaggctccattcttaccgtctcccgaacaccatgtgacccagacctgccatacatctgcacagcccagaaccccgtcagccagagaagctccctccctgtccatgttgggcagttctgtacagatccaggagcctccagaggaggaacaacgggggagactgtggtaggggtcctgggagagccagtcaccctgccacttgcactcccagcctgccgggacacagagaaggttgtctggttgtttaacacatccatcattagcaaagagagggaagaagcagcaacggcagatccactcattaaatccagggatccttacaagaacagggtgtgggtctccagccaggactgctccctgaagatcagccagctgaagatagaggacgccggcccctaccatgcctacgtgtgctcagaggcctccagcgtcaccagcatgacacatgtcaccctgctcatctaccgcaggctgaggaagcccaaaatcacgtggagcctcaggcacagtgaggatggcatctgcaggatcagcctgacctgctccgtggaggacgggggaaacactgtcatgtacacatggaccccgctgcagaaggaagctgttgtgtcccaaggggaatcacacctcaatgtctcatggagaagcagtgaaaatcaccccaacctcacatgcacagccagcaaccctgtcagcaggagttcccaccagtttctttctgagaacatctgttcaggacctgagagaaacacaaagctttggattgggttgttcctgatggtttgccttctgtgcgttgggatcttcagctggtgcatttggaagcgaaaaggacggtgttcagtcccagccttctgttccagccaagctgaggccccagcggatacaccaggatatgagaagctggacactcccctcaggcctgccaggcaacagcctacacccacctcagacagcagctctgacagcaacctcacaactgaggaggatgaggacaggcctgaggtgcacaagcccatcagtggaagatatgaggtgtttgaccaggtcactcaggagggcgctggacatgacccagcccctgagggccaagcagactatgatcccgtcactccatatgtcacggaagttgagtctgtggttggagagaacaccgtgtatgcacaagtgttcaacttacagggaaagaccccagtttctcagaaggaagagagctcagccacaatctactgctccatacggaaacctcaggtggtgccaccaccacaacagaatgatcttgagattcctgaaagtcctacctatgaaaatttcacctgaaaggaaaagcagctgctgcctctctcctgggaccgtggggttggaaagtcagctggacctcatggggcctggggctcgctgacagaagcacctcagaatttccttcagtgcctcagagatgcctggatgtggcccctccccctccttctcacccttaaggactcccaaacccattaatagttcagacacaggctccttcttggagcctatgggcttcagatgtctttgccccatttgtcacctcgcacacttatagcgtttcctcctcgaaattctaccaagactggtcaaatgttgctgaggggcctggaccagctgtcctttacaccaccttctcaacactgctgaaaagaaccccaagagaattgtcacacatgacacaagatgtacataatatcatgctcacgcctggagtgttatttaaaataaaaggcaggaaataaaaaaaaaaaa

SEQ ID NO: 13 - protein, 243 aa, Homo sapien [sequence omitted]

SEQ ID NO: 14 - DNA, 1048 bp, Homo sapien
ctgtgaaagaaggaagcatgtggtccagaggttgggattcgtgtctggctctggaattgctactgctgcctctgtcactcctggtgaccagcattcaaggtcacttggtacatatgaccgtggtctccggcagcaacgtgactctgaacatctctgagagcctgcctgagaactacaaacaactaacctggttttatactttcgaccagaagattgtagaatgggattccagaaaatctaagtactttgaatccaaatttaaaggcagggtcagacttgatcctcagagtggcgcactgtacatctctaaggtccagaaagaggacaacagcacctacatcatgagggtgttgaaaaagactgggaatgagcaagaatggaagatcaagctgcaagtgcttgaccctgtacccaagcctgtcatcaaaattgagaagatagaagacatggatgacaactgttatctgaaactgtcatgtgtgatacctggcgagtctgtaaactacacctggtatggggacaaaaggcccttcccaaaggagctccagaacagtgtgcttgaaaccacccttatgccacataattactccaggtgttatacttgccaagtcagcaattctgtgagcagcaagaatggcaccgtctgcctcagtccaccctgtaccctggcccggtcctttggagtagaatggattgcaagttggctagtggtcacggtgcccaccattcttggcctgttacttacctgagatgagctcttttaactcaagcgaaacttcaaggccagaagatcttgcctgttggtgatcatgctcctcagcaggacagagactgtataggctgaccagaagcatgctgctgaattatcaacgaggattttcaagttaacttttaaatactggttattatttaattttatatccctttgttgttttgtagtacacagagattatagagatacacatgcttttttcccaaaattgtgacaacattatgtggaatcttttattatttttaaaataaaaagatataattataaaaaaaaaaa

SEQ ID NO: 15 - protein, 240 aa, Mus musculus
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSSGVCWTATWLVVTTLIIHRILLT

SEQ ID NO: 16 - DNA, 1154 bp, Mus musculus
acttctcttacagttgtctccagtgttctggggaagcttctctaagtattatgtgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcgtcgactgattcctgtcacctgaggctatcgtgtgaggtaaaggaccagcatgttgactatacttggtatgagagctcgggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatctagccagatcttctggagtatgttggactgcaacttggctagtggtcacaacactcatcattcacaggatcctgttaacctgacaagaactcttctcacccaagaaggcaacttggaagcacagagtcttgccttcatccctagcagtgttcctagccagcgaacaactctggctctatttggacaaaggaaaatgtgtttactgaacgtctgcgagagtttgcatgcatgctctatgaaacaagcacaggaccttgtacagtgctccaccactgacctgtgtgcccagtcctttacaaagatttcaaatcaaccttttaaaaactgtgcataatatctaattttatataccctagttgtttcccaacatatattaaagataaatgcattctttttaccaaaatgtgactatattattttcatgttttcatatctctttttaaaataaattcttttaaaaaact

SEQ ID NO: 17 - DNA, 20 bp, Homo sapien
aaaaaccaagccacaactgg

SEQ ID NO: 18 - DNA, 20 bp, Homo sapien
atttcctctttgccctttgg

SEQ ID NO: 19 - DNA, 180 bp, Homo sapien
aaaaaccaagccacaactgggagcagatatttacagtatatgtaactgacaaaggactaatatccagaaacatgtaatgaattcctgtgaacacacacacacacacacacacacacacacacagagagagagagagagggagacacatgaacagcatttcccaaagggcaaagaggaaat

SEQ ID NO: 20 - DNA, 20 bp, Homo sapien
tccatccttccagctcagtc

SEQ ID NO: 21 - DNA, 21 bp, Homo sapien
tcccatttaccctgtgtgatt

SEQ ID NO: 22 - DNA, 235 bp, Homo sapien
tccatccttccagctcagtctccttgttgtgtgtgtgttttctcctctaatttttaacgtctgtgggtacatagtagatatacatatatgtgtgtgtgtgtgtgtgtgtgtgtgtgtgtgtgtgtgtgtgtgtatgtatatatatatgtatggggtatgcatatatttgtatggggtatatgagatatgttgatagaggcatgcaatatgtaataatcacacagggtaaatggga

SEQ ID NO: 23 - DNA, 20 bp, Homo sapien
acactggagcacccaaattc

SEQ ID NO: 24 - DNA, 20 bp, Homo sapien
ggagtgctgaagtcctccac

SEQ ID NO: 25 - DNA, 154 bp, Homo sapien
acactggagcacccaaattcataaagcaaatattaccagagagagagtgagagtaagagtgtgtgtgtgtgtgtgtgtgtgtgtgtgtgtgtgtgtgtgtgagacagagagagagagactgcaacagaatagtagtggaggacttcagcactcc

SEQ ID NO: 26 - DNA, 19 bp, Homo sapien
tcagttccataggctgacg

SEQ ID NO: 27 - DNA, 18 bp, Homo sapien
cattgctgatgctggagg

SEQ ID NO: 28 - DNA, 255 bp, Homo sapien
  modified_base (116 and 205): N = A, C, G, or T
tcagttccataggctgacgaaacacaaagttcaggctactggctttgcttcttatcctagtattagagtgatttctccagtggttcctagtgtcgatatcataaaccttgaatgantcaatctgtctcaaacacacacatacacacatacacacatacacacacacacacacacacacacactcctgcacagagggttctcagtnaccataagtcactcagagtggagctgctccttcctccagcatcagcaatg

SEQ ID NO: 29 - DNA, 16 bp, Homo sapien
tagcagatcccccgtc

SEQ ID NO: 30 - DNA, 23 bp, Homo sapien
tgaatcctacccctaagtagaat

SEQ ID NO: 31 - DNA, 241 bp, Homo sapien
agctgacatagcagatcccccgtcaccaagttgcgtgcacacgatgcatacacacacacacgcatgcggcaagcacacacacacacacacacacacacacacacacacacttctctttcttgctttgtattatagatgagattctacttaggggtaggattcattattcatgaagggtgtggtcaggtgaggcatgttggaagcaaaatgcgaattaggtaaggtggagtagaagagagct

SEQ ID NO: 32 - DNA, 20 bp, Homo sapien
acatgaacctgggaggtgag

SEQ ID NO: 33 - DNA, 20 bp, Homo sapien
accatgcctggctaatttgt

SEQ ID NO: 34 - DNA, 160 bp, Homo sapien
acatgaacctgggaggtgaggttgcagtgagtgaaaatcatgctactgcacttcagcctgagtgacagagggagactgtctcaaaaaaaaaaaaaaaaaaacatgtgcacacacacacacacacacacacacacacacacacaaattagccaggcatggt

SEQ ID NO: 35 - DNA, 20 bp, Homo sapien
cccatatgctgcttccagat

SEQ ID NO: 36 - DNA, 20 bp, Homo sapien
aagggtgtgtgcatgtgtgt

SEQ ID NO: 37 - DNA, 266 bp, Homo sapien
acacacatgcacacaccctttaggattttattggaattacattgaatcttggtcgtttacctctacacatacacacacacacacacacacacacactgatatccagtagagcaaggcttctcactttgctcttcttttttggttatcttgtgcccataccacattgttccaaatattttagattgataaaatgtctcccttcccaaaggtaattactggcctgactgtgatggaaatcagcacttccccatatgctgcttccagat

(19) United States
(12) Patent Application Publication
(10) Pub. No.: US 2003/0054002 A1
(43) Pub. Date: Mar. 20, 2003

(54) Isolation of genes within SLE-1B that mediate a break in immune tolerance

(75) Inventors: Ward Wakeland, Dallas, TX (US); Amy Wandstrat, Dallas, TX (US); Laurence C. Morel, Gainsville, FL (US)

Correspondence Address: Steven L. Highlander, FULBRIGHT & JAWORSKI L.L.P., Suite 2400, 600 Congress Avenue, Austin, TX, 78701, US

(73) Assignee: Board of Regents, The University of Texas System

(21) Appl. No.: 09/860,836

(22) Filed: May 17, 2001

Related U.S. Application Data
(60) Provisional application No. 60/204,963, filed on May 17, 2000.
(60) Provisional application No. 60/234,457, filed on Sep. 21, 2000.

(51) Int. Cl. A61K 39/395; C12Q 1/68; C12P 19/34; C12M 1/34

(52) U.S. Cl. 424/130.1; 435/6; 435/91.2; 435/287.2

(57) ABSTRACT

The invention relates to the determination of a genetic loci and genes therein which are related to an increased susceptibility to the development of systemic autoimmunity, specifically, systemic autoimmune erythematosus. The loci and genes were isolated utilizing a strategy based on the generation and phenotypic analysis of congenic recombinants to identify SLE susceptibility genes in NZM2410 mice. The elucidated genes facilitate screening for susceptibility as well as use in therapeutic and prophylactic roles. In addition, the murine model may be used in the screening of compounds which may be of therapeutic or prophylactic benefit in the treatment of systemic autoimmune disorders.

This application claims the benefit of priority of U.S. Provisional Applications Nos.60/204,963, filed May 17, 2000, and 60/234,457, filed Sep. 21, 2000, the entire content of each being incorporated by reference.

The government owns rights in the present invention pursuant to a grant from the National Institute of Health.

### BACKGROUND OF THE INVENTION

1. Field of the Invention

The present invention relates generally to the fields of molecular biology and diagnostics. More particularly, it concerns the identification of genetic loci and genes involved in the breakdown of immune tolerance leading to the development of systemic autoimmunity, particularly systemic lupus erythematosus.

2. Description of Related Art

Systemic lupus erythematosus (SLE) is mediated by a complex interaction of genetic and environmental elements. Although the crucial role of genetic predisposition in susceptibility to SLE has been known for decades, only minimal progress has been made towards elucidating the specific genes involved in human disease. Recently, several groups have reported linkage analyses that provide estimates of the number and chromosomal locations of at least some of the susceptibility genes for human SLE. These studies have provided interesting insights into the complexity of the genetic interactions involved in SLE, but have not resulted in the identification of specific genes or genetic pathways involved in disease pathogenesis. This reflects the difficulties inherent in the analysis of complex genetic diseases, coupled with the absence of sample repositories of sufficient size to allow definitive genetic analysis.

Genetic analyses of lupus susceptibility in the mouse have progressed significantly over the last 5 years, predominantly due to the availability of unique and powerful experimental genetic tools. The chromosomal locations of genes mediating susceptibility in the NZB/W, MRL/1pr, and BXSB mouse models have been determined via genome scans. These studies have demonstrated that susceptibility to lupus in the mouse is inherited in a complex fashion that is quite similar to human SLE, involving both genetic interactions and additive effects of individual genes.

Despite the advances that have occurred in understanding the genetic factors that create a predisposition for the disease, the specific genes responsible for the development of the disease have previously remained unknown.

### SUMMARY OF THE INVENTION

The instant invention addresses the deficiencies in the art by setting forth a genetic loci and four genes within this loci which are associated with an increased susceptibility to the development of systemic autoimmunity, specifically systemic lupus erythematosus. The present invention thus relates methods for the diagnosis, prevention and treatment of various forms of systemic autoimmunity, with specific emphasis on systemic lupus erythematosus.

In one aspect, the invention relates to methods of detecting an increased susceptibility to systemic autoimmunity. A specific embodiment of the instant invention therefore relates to a method of screening for susceptibility to a systemic autoimmune disorder comprising screening for at least one mutation within the SLE-1B loci. It is contemplated that the invention will be particularly useful in the detection of a susceptibility to systemic lupus erythematosus.

A person of ordinary skill would recognize a variety of methods that alterations in the SLE-1B loci might be readily detected or assayed. Specific assays and screening methods for alterations in genetic sequence or an encoded peptide are well within the purview of a person of ordinary skill. It is nevertheless contemplated that certain screening techniques will be of use. In various embodiments of the instant invention, the method of screening will comprise FISH, the use of a DNA array, PCR amplification or hybridization with a polynucleotide probe. Where detection is by polynucleotide hybridization, it is further contemplated that detection may be facilitated by the techniques of either Northern or Southern blotting.

In a particular embodiment of the invention where a DNA array is employed, it is contemplated that the DNA array may be arranged on a DNA or gene chip. The DNA array may be further characterized as a DNA array comprising polynucleotide molecules complementary to genetic sequence within the SLE-1B loci, wherein the polynucleotide molecules comprise mutant sequence indicative of a systemic autoimmune phenotype. In a further embodiment, the DNA array is characterized as a DNA array for use in the detection of genetic susceptibility to systemic lupus erythematosus comprising nucleic acid probes comprising sequence complementary to one or more mutated regions of genes selected from Cd48, Cd84, Slam, 2B4, Ly108, Cs1, Dedd, Nit1, Ly9, Usf1 and/or Golga4.

The genetic alteration giving rise to a phenotype with increased susceptibility to the development of autoimmunity may occur anywhere within the SLE-1B loci. Nevertheless, in one embodiment of the instant invention, the mutation or mutations giving rise to the phenotype is within the coding sequence of a gene or genes within the SLE-1B loci. In alternate embodiments of the invention, the mutation occurs in one or more of the genes encoding CD48, CD84, SLAM, 2B4, LY108, CS1, DEDD, NIT1, LY9, USF1 and/or GOLGA4.

The method of screening may be further characterized as a means of screening for susceptibility of a host to systemic lupus erythematosus comprising obtaining a tissue sample from the host, isolating nucleic acid from the sample and assaying for the presence of at least one mutation within the SLE-1B loci. Detection of a mutation in this context is indicative of an increased susceptibility to developing systemic lupus erythematosus. In a particular embodiment of this method, the host to be screened will be a human.

While methods of diagnosing systemic autoimmunity and systemic lupus erythematosus are important aspects of the invention, the invention also relates to methods of preventing the development of systemic autoimmunity as well as treating such disorders once developed. A specific embodiment of the instant invention therefore comprises a method of treating a systemic autoimmune disorder comprising administering to a host a construct comprising wild-type sequence from within the SLE-1B loci. It is specifically contemplated that this method will be specifically applicable in the treatment of systemic lupus erythematosus.

In the context of treating or preventing systemic autoimmunity, it is contemplated that a variety sequence constructs from within the SLE-1B loci may be administered. Nevertheless, in certain embodiments of the invention, the delivered construct will comprise sequence encoding CD48, CD84, SLAM, 2B4, LY108, CS1, DEDD, NIT1, LY9, USF1 and/or GOLGA4.

The methods and compositions of the instant invention further provide for a means of screening for potential therapeutic compounds for use in the prevention or treatment of systemic autoimmunity, particularly systemic lupus erythematosus. In a particular embodiment, the method of screening for therapeutics for treating systemic autoimmunity comprises a mouse with at least one mutation within the SLE1-B loci, wherein said mouse develops an autoimmune phenotype. The method may be further characterized as comprising administering to the mouse a compound and monitoring the progression of the autoimmune phenotype in the mouse. In another embodiment, this method is useful in screening for compounds for the treatment of prevention of systemic lupus erythematosus. In an alternate embodiment, the mutation giving rise to the SLE phenotype within the subject mouse arises in the genetic sequence encoding CD48, CD84, SLAM, 2B4, LY108, CS1, DEDD, NIT1, LY9, USF1 and/or GOLGA4

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Congenic dissection of lupus susceptibility in the NZM2410 mouse

FIG. 2: Linkage map illustrating the strong homology between the Sle1 gene cluster on murine chromosome 1 and the 1q23--1

q42 region on human chromosome 1.

FIG. 3: Recombinational breakpoints in congenic recombinant intervals used to define Sle1a, Sle1b, and Sle1c. Heavy line indicates NZM-derived genome, light line indicates position of B65.NZM junction.

FIG. 4: Pedigree of recombinant chromosomes leading to the production of B6.NZMc1a and B6.NZMc1b FIG. 5: Subnucleosome of autoantibodies spontaneously produced by 10 mice of each strain at 7 months of age.

FIG. 6: YAC and BAC contig for the Sle1b critical interval

FIG. 7: Tiling path of 7 overlapping BAC clones that span the critical region for Sle1b. A physical map of this segment is shown below, together with the most interesting positional candidate genes identified

FIG. 8: YAC and BAC contig of the Sle1a minimal susceptibility region. YACs are shown above in yellow and BACs below in blue. BAC markers derived from end-sequence are indicated by squares while circles indicate genes and MIT markers.

FIG. 9: Development of fatal lupus for various congenic sub-intervals of the gene cluster crossed to NZW.

FIG. 10: Positions of sub-congenic intervals in B6.NZMc7c and B6.NZMc7t

FIG. 11: Regression analysis of gene expression patterns in splenic CD4 T cells (left) and B cells (right) from NZM2410 (y axis) versus B6 (x axis).

FIG. 12: Penetrance of autoantibody production by Sle1a is allele does dependent.

FIG. 13: Penetrance of autoantibody production by Sle1b is allele does dependent

FIG. 14: Breeding strategy for BAC rescue test.

FIG. 15: Four susceptibility genes detected within the Sle1 congenic interval

FIG. 16: Syntenic relationship of murine chromosome 1 and human 1q2

FIG. 17: Genetic and phenotypic properties of the congenic intervals in B6.Sle1a and B6.sle1b mice.

FIG. 18: Positional cloning of Sle1b

FIG. 19: Genomic organization and microsatelitte loci detected in the CD2 gene cluster of human 1q21.

### DESCRIPTION OF ILLUSTRATIVE EMBODIMENTS

Genes within the Sle1 gene cluster play an essential role in disease pathogenesis in both human and mouse systemic autoimmunity. The present invention sets forth the identities of the genes within the SLE-1B cluster involved in the development of SLE. The identification of these genes in mouse and subsequently in humans provides vital insights into the genetic mechanisms responsible for dysregulating a major biological pathway initiating systemic autoimmunity, specifically, lupus pathogenesis.

The identification of the genes within the gene cluster responsible for an increased susceptibility to developing systemic autoimmunity facilitates not only the diagnosis of individuals with a predilection for developing an autoimmune phenotype, but also establishes a means of inhibiting the development of the phenotype and treating those currently suffering from an autoimmune disorder. Further, mouse models, disclosed herein are of use in screening potential therapeutic compounds.

The instant invention relates a means of screening for an increased susceptibility to developing an autoimmune phenotype. Detecting specific genetic changes within the loci set forth herein, specifically within the genes encoding CD48, CD84, SLAM, 2B4, LY108, CS1, DEDD, NIT1, LY9, USF1 and/or GOLGA4, indicates that the host from which the sample was derived will exhibit an increased susceptibility to the development of a systemic immune disorder, e.g., systemic lupus erythematosus. Means for detecting genetic alterations are well known in the art. Traditional methods, including PCR, sequencing, hybridization and FISH are well within the skill of the art. The recent advances within the field of DNA arrays and gene chips further facilitates the rapid detection of genetic alterations. Gene chips allow for the detection of a wide range of genetic alterations from single nucleotide polymorphisms (SNP) to a loss of heterozygocity (LOH). In the context of the instant invention, it is contemplated that gene chips will be particularly useful in screening for the specific alterations indicative of an increased susceptibility to systemic autoimmunity.

In addition, the loci and genes set forth herein provide a means of preventing or treating systemic autoimmunity. An awareness of the specific alterations that lead to the breakdown in the immune system ultimately leading to the development of a systemic autoimmune phenotype facilitates the substitution of either functional genes or proteins to remedy the extant defects. Therefore, the instant invention comprises a means of administering to a host organism wild-type genes or wild-type protein to offset the altered gene or protein leading to the development of autoimmunity. A person of ordinary skill would readily recognize techniques by which genes and proteins may be administered to correct such defects. For example, gene therapy allows for the replacement of a defective gene with a wild-type sequence, which facilitates the production of a functional protein. In mice, the Sle1 gene cluster can be suppressed by genetic modifiers and this suppression completely inhibits the development of disease in mice genetically programmed to die of systemic autoimmunity.

The instant invention also discloses a murine model system useful in the screening of compounds that are prophylactic or therapeutic for systemic autoimmunity, specifically, systemic lupus erythematosus.

A. Nucleic Acids

The instant invention sets forth genetic loci and specific genes responsible for an increased susceptibility to developing systemic autoimmunity. Therefore, the use, manipulation, detection, isolation, amplification and screening of nucleic acids are important aspects of the invention.

In the context of the instant invention, genes are sequences of DNA in an organism's genome encoding information that is converted into various products making up a whole cell. They are expressed by the process of transcription, which involves copying the sequence of DNA into RNA. Most genes encode information to make proteins, but some encode RNAs involved in other processes. If a gene encodes a protein, its transcription product is called mRNA (“messenger” RNA). After transcription in the nucleus (where DNA is located), the mRNA must be transported into the cytoplasm for the process of translation, which converts the code of the mRNA into a sequence of amino acids to form protein. In order to direct transport into the cytoplasm, the 3′ ends of mRNA molecules are post-transcriptionally modified by addition of several adenylate residues to form the “polyA” tail. This characteristic modification distinguishes gene expression products destined to make protein from other molecules in the cell, and thereby provides one means for detecting and monitoring the gene expression activities of a cell.

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double stranded nucleic acid by the prefix “ds,” and a triple stranded nucleic acid by the prefix “ts.”

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 8 9%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

Hybridization is understood to mean the forming of a double-stranded molecule and/or a molecule with partial double-stranded nature. Stringent conditions are those that allow hybridization between two homologous nucleic acid sequences, but precludes hybridization of random sequences. For example, hybridization at low temperature and/or high ionic strength is termed low stringency. Hybridization at high temperature and/or low ionic strength is termed high stringency. Low stringency is generally performed at 0.15 M to 0.9 M NaCl at a temperature range of 20° C. to 50° C. High stringency is generally performed at 0.02 M to 0.15 M NaCl at a temperature range of 50° C. to 70° C. It is understood that the temperature and/or ionic strength of a desired stringency are determined in part by the length of the particular probe, the length and/or base content of the target sequences, and/or to the presence of formamide, tetramethylammonium chloride and/or other solvents in the hybridization mixture. It is also understood that these ranges are mentioned by way of example only, and that the desired stringency for a particular hybridization reaction is often determined empirically by comparison to positive and/or negative controls.

Accordingly, the nucleotide sequences of the disclosure may be used for their ability to selectively form duplex molecules with complementary stretches of genes or RNA. Depending on the application envisioned, it is preferred to employ varying conditions of hybridization to achieve varying degrees of selectivity of probe towards target sequence.

Nucleic acid molecules having sequence regions consisting of contiguous nucleotide stretches of about 13, 14, 15, 16, 17, 18, 20, 25, 30, 35, 40, 45, 50, 55, 60, 70, 80, 90, 100, 125, 150, 175, 200, 250, 300, 350, 400 or more basepairs (bp) to about 5000 bp, or even up to and including sequences of about 30-50 cM or so, identical or complementary to the target DNA sequence, are particularly contemplated as hybridization probes for use in embodiments of the instant invention. It is contemplated that long contiguous sequence regions may be utilized including those sequences comprising about 100, 200, 300, 400, 500, 600, 700, 800, 900, 1000, 1100, 1200, 1300, 1400, 1500, 2000, 2500, 3000, 3500, 4000, 4500, 5000, 5500, 6000, 6500, 7000, 7500, 8000, 8500, 9000, 9500, 10,000 or more contiguous nucleotides or up to and including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more cM.

Preferred embodiments of the instant invention involve the detection of genetic changes in an individual by the ability of host chromosomal DNA to hybridize to a specific probe. In a preferred embodiment of the instant invention, probes constitute single stranded DNA of from 18 b.p. to 1000 base pair up to and including 1 cM. It is envisioned that probes may constitute, for example, synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change which may be indicative of an increased susceptibility to SLE in a host. An example of a change detectable by the failure of a probe to hybridize to a hosts chromosomal DNA is a mutation such as, for example, a single nucleotide polymorphism.

B. Screening

The genetic mutations or changes indicating an increased susceptibility to SLE are detectable by a variety of methods, that may be utilized to identify those a host or a host's cells exhibiting mutations at one or more selected loci in genes identified herein. The following description sets forth techniques which are exemplary of means a person of ordinary skill would employ in the detection of the disclosed genetic alterations.

1. Gene Chips and DNA Arrays

DNA arrays and gene chip technology provides a means of rapidly screening a large number of DNA samples for their ability to hybridize to a variety of single stranded DNA probes immobilized on a solid substrate. Specifically contemplated are chip-based DNA technologies such as those described by Hacia et al. (1996) and Shoemaker et al. (1996). These techniques involve quantitative methods for analyzing large numbers of genes rapidly and accurately. The technology capitalizes on the complementary binding properties of single stranded DNA to screen DNA samples by hybridization. Pease et al. (1994); Fodor et al. (1991). Basically, a DNA array or gene chip consists of a solid substrate upon which an array of single stranded DNA molecules have been attached. For screening, the chip or array is contacted with a single stranded DNA sample which is allowed to hybridize under stringent conditions. The chip or array is then scanned to determine which probes have hybridized. In a particular embodiment of the instant invention, a gene chip or DNA array would comprise probes specific for chromosomal changes evidencing the development of a neoplastic or preneoplastic phenotype. In the context of this embodiment, such probes could include synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832 which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal, for example, luminescence. The target nucleic acid may be immobilized onto the integrated microchip that also supports a phototransducer and related detection circuitry. Alternatively, a gene probe may be immobilized onto a membrane or filter which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellous membranes, the DNA probes are spotted onto the membranes. A UV light source (Stratalinker,™ Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BINDTM Costar, Cambridge, Mass.).

2. Fluorescent In Situ Hybridization

As described in U.S. Pat. Nos. 5,427,910 and 5,523,207, which are expressly incorporated by reference, flourescent in situ hybridization (FISH) involves the introduction of a nucleic acid probe with a defined nucleotide sequence into a cell, where it preferentially hybridizes with a specific complementary nucleotide sequence of DNA, or target DNA, on one or more chromosomes within the cell. The target nucleotide sequence may be unique or repetitive, as long as it can be used to distinguish one or more specific chromosomes. The probe is labeled with a fluorescent tag so that cells with the target DNA sequence(s), to which the marked probes hybridize, can be detected microscopically. Each chromosome containing the targeted DNA sequence, and hence the hybridized probe, will emit a fluorescent signal or spot. Thus, for example, specimens hybridized with a DNA sequence known to be contained on chromosome number 21 will produce two fluorescent spots in cells from normal patients and three spots from Down's Syndrome patients because they have an extra chromosome number 21.

3. Polymerase Chain Reaction

The technique of “polymerase chain reaction,” or “PCR,” as used herein generally refers to a procedure wherein minute amounts of a specific piece of nucleic acid, RNA and/or DNA, are amplified as described in U.S. Pat. Nos. 4,683,195, 4,683,202, and 4,683,194, which are herein expressly incorporated by reference. Generally, sequence information from the ends of the region of interest or beyond needs to be available, such that oligonucleotide primers can be designed. These primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid that is complementary to a particular nucleic acid.

4. Northern and Southern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

5. Restriction Fragment Length Polymorphism

“Restriction Enzyme Digestion” of DNA refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 μg of plasmid or DNA fragment is used with about 1-2 units of enzyme in about 20 μl of buffer solution. Appropriate buffers and substrate amounts for particular restriction enzymes are specified by the manufacturer. Incubation of about 1 hour at 37° C. is ordinarily used, but may vary in accordance with the supplier's instructions.

Restriction fragment length polymorphisms (RFLPs) analysis capitalizes on the selectivity of restriction enzymes to detect the genetic changes in specific loci. RFLP are genetic differences detectable by DNA fragment lengths, typically revealed by agarose gel electrophoresis, after restriction endonuclease digestion of DNA. There are large numbers of restriction endonucleases available, characterized by their nucleotide cleavage sites and their source, e.g., Eco RI. Variations in RFLPs result from nucleotide base pair differences which alter the cleavage sites of the restriction endonucleases, yielding different sized fragments. Means for performing RFLP analyses are well known in the art.

As described in U.S. Pat. No. 5,580,729, expressly incorporated by reference, one means of testing for loss of an allele is by digesting the first and second DNA samples of the neoplastic and non-neoplastic tissues, respectively, with a restriction endonuclease. Restriction endonucleases are well known in the art. Because they cleave DNA at specific sequences, they can be used to form a discrete set of DNA fragments from each DNA sample. The restriction fragments of each DNA sample can be separated by any means known in the art. For example, an electrophoretic gel matrix can be employed, such as agarose or polyacrylamide, to electrophoretically separate fragments according to physical properties such as size. The restriction fragments can be hybridized to nucleic acid probes which detect restriction fragment length polymorphisms, as described above. Upon hybridization hybrid duplexes are formed which comprise at least a single strand of probe and a single strand of the corresponding restriction fragment. Various hybridization techniques are known in the art, including both liquid and solid phase techniques. One particularly useful method employs transferring the separated fragments from an electrophoretic gel matrix to a solid support such as nylon or filter paper so that the fragments retain the relative orientation which they had on the electrophoretic gel matrix. The hybrid duplexes can be detected by any means known in the art, for example, the hybrid duplexes can be detected by autoradiography if the nucleic acid probes have been radioactively labeled. Other labeling and detection means are known in the art and may be used in the practice of the present invention.

Nucleic acid probes which detect restriction fragment length polymorphisms for most non-acrocentric chromosome arms are available from the American Type Culture Collection (Rockville, Md.). These are described in the NIH Repository of Human DNA Probes and Libraries, published in August, 1988. Methods of obtaining other probes which detect restriction fragment length polymorphisms are known in the art. The statistical information provided by using the complete set of probes which hybridizes to each of the non-acrocentric arms of the human genome is useful prognostically. Other subsets of this complete set can be used which also will provide useful prognostic information. Other subsets can be tested to see if their use leads to measures of the extent of genetic change which correlates with prognosis, as does the use of the complete set of alleles.

C. Gene Therapy

Mutations or alterations in the genetic sequence of SLE-1B results in an increased susceptibility to systemic autoimmunity. Therefore, if the mutated genes can be replaced or altered to express wild-type phenotype, it may ultimately prevent development of the disease or provide some therapeutic benefit in the event that a disease state has developed. It is therefore specifically contemplated that the genes set forth in the instant invention are useful in the treatment and prevention of systemic autoimmunity, specifically, systemic lupus erythromatosus. The general approach to this aspect of the present invention concerning systemic autoimmunity is to provide a cell with a wild-type CD48, CD84, SLAM, 2B4, LY108, CS1, DEDD, NITI, LY9, USF1 and/or GOLGA4 protein, thereby permitting the proper regulatory activity of the proteins to take effect. While it is conceivable that the protein may be delivered directly, a preferred embodiment involves providing a nucleic acid encoding a a wild-type CD48, CD84, SLAM, 2B4, LY108, CS1, DEDD, NIT1, LY9, USF1 and/or GOLGA4 to the cell. Following this provision, the polypeptide is synthesized by the transcriptional and translational machinery of the cell, as well as any that may be provided by the expression construct. All such approaches are herein encompassed within the term “gene therapy”.

In certain embodiments of the invention, the nucleic acid encoding the gene may be stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

1. DNA Delivery Using Viral Vectors

The ability of certain viruses to infect cells or enter cells via receptor-mediated endocytosis, and to integrate into host cell genome and express viral genes stably and efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

a. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

The expression vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other marnmalian species that are permissive for human adenovirus. Such cells include, e.g., Vero cells or other monkey embryonic mesenchymal or epithelial cells. As stated above, the preferred helper cell line is 293.

Racher et al. (1995) discloses improved methods for culturing 293 cells and propagating adenovirus. In one fornat, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another fornat, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1991; Rich et al, 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing human primary cells.

b. AAV Vectors

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al, 1988; McLaughlin et al., 1988). Details concerning the generation and use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al (1993); and Walsh et al (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hennonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM⁴5 (McCarty et al, 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

C. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al, 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

d. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al, 1990). This suggested that large portions of the genome could be replaced with foreign genetic material. Chang et al recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al, 1991).

In certain further embodiments, the gene therapy vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

e. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Other Methods of DNA Delivery

In various embodiments of the invention, DNA is delivered to a cell as an expression construct. In order to effect expression of a gene construct, the expression construct must be delivered into a cell. As described herein, the preferred mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA or plasmids. Transfer of the construct may be performed by any of the methods mentioned which physically or chemically permeabilize the cell membrane. Some of these techniques may be successfully adapted for in vivo or ex vivo use, as discussed below.

a. Liposome-Mediated Transfection

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

b. Electroporation

In certain embodiments of the present invention, the expression construct is introduced into the cell via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

C. Calcium Phosphate Precipitation or DEAE-Dextran Treatment

In other embodiments of the present invention, the expression construct is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

d. Particle Bombardment

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

e. Direct Microinjection or Sonication Loading

Further embodiments of the present invention include the introduction of the expression construct by direct microinjection or sonication loading. Direct microinjection has been used to introduce nucleic acid constructs into Xenopus oocytes (Harland and Weintraub, 1985), and LTK fibroblasts have been transfected with the thymidine kinase gene by sonication loading (Fechheimer et al., 1987).

f. Adenoviral Assisted Transfection

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994).

g. Receptor Mediated Transfection

Still further expression constructs that may be employed to deliver the tissue-specific promoter and transforming construct to the target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Specific delivery in the context of another mammalian cell type is described by Wu and Wu (1993).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. In the context of the present invention, the ligand will be chosen to correspond to a receptor specifically expressed on the neuroendocrine target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al (1987) employed lactosyl-ceramide, a galactose-terminal asialganglioside, incorporated into liposomes and observed an increase in the uptake of the insulin gene by hepatocytes. It is contemplated that the tissue-specific transforming constructs of the present invention can be specifically delivered into the target cells in a similar manner.

3. Antisense

In alternative embodiments, CD48, CD84, SLAM, 2B4, LY108, CS1, DEDD, NIT1, LY9, USF1 and/or GOLGA4 encoding nucleic acids employed may actually encode antisense constructs that hybridize, under intracellular conditions, to a mutant CD48, CD84, SLAM, 2B4, LY108, CS1, DEDD, NITI, LY9, USF1 and/or GOLGA4 encoding nucleic acids. The term “antisense construct” is intended to refer to nucleic acids, preferably oligonucleotides, that are complementary to the base sequences of a target DNA or RNA. Targeting double-stranded (ds) DNA with an antisense construct leads to triple-helix formation; targeting RNA will lead to double-helix formation. Antisense nucleic acids, when introduced into a target cell, specifically bind to their target polynucleotide, for example a mutant Cd48, Cd84, Slam, 2B4, Ly108, Cs1, Dedd, Nit1, Ly9, Usf1 and/or Golga4 gene and interfere with transcription, RNA processing, transport, translation and/or stability. Antisense RNA constructs, or DNA encoding such antisense RNAs, may be employed to inhibit mutant Cd48, Cd84, Slam, 2B4, Ly108, Cs1, Dedd, Nit1, Ly9, Usf1 and/or Golga4 gene transcription or translation or both within the cells of the present invention.

Antisense constructs may be designed to bind to the promoter and other control regions, exons, introns or even exon-intron boundaries of a gene. Antisense RNA constructs, or DNA encoding such antisense RNA's, may be employed to inhibit gene transcription or translation or both within a host cell, either in vitro or in vivo, such as within a host animal, including a human subject. Nucleic acid sequences which comprise “complementary nucleotides” are those which are capable of base-pairing according to the standard Watson-Crick complementarity rules. That is, that the larger purines will base pair with the smaller pyrimidines to form combinations of guanine paired with cytosine (G:C) and adenine paired with either thymine (A:T), in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

As used herein, the term “complementary” means nucleic acid sequences that are substantially complementary over their entire length and have very few base mismatches. For example, nucleic acid sequences of fifteen bases in length may be termed complementary when they have a complementary nucleotide at thirteen or fourteen positions with only a single mismatch. Naturally, nucleic acid sequences which are “completely complementary” will be nucleic acid sequences which are entirely complementary throughout their entire length and have no base mismatches.

Other sequences with lower degrees of homology also are contemplated. For example, an antisense construct which has limited regions of high homology, but also contains a non-homologous region (e.g., a ribozyme) could be designed. These molecules, though having less than 50% homology, would bind to target sequences under appropriate conditions.

While all or part of the Cd48, Cd84, Slam, 2B4, Ly108, Cs1, Dedd, Nit1, Ly9, Usf1 and/or Golga4 gene sequences may be employed in the context of antisense construction, short oligonucleotides are easier to make and increase in vivo accessibility. However, both binding affinity and sequence specificity of an antisense oligonucleotide to its complementary target increases with increasing length. One can readily determine whether a given antisense nucleic acid is effective at targeting of the corresponding host cell gene simply by testing the constructs in vitro to determine whether the function of the endogenous gene is affected or whether the expression of related genes having complementary sequences is affected.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression.

4. Ribozymes

Another method for inhibiting mutant CD48, CD84, SLAM, 2B4, LY108, CS1, DEDD, NIT1, LY9, USF1 and/or GOLGA4 expression contemplated in the present invention is via ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al, 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al, 1992). Recently, it was reported that ribozymes elicited genetic changes in some cells lines to which they were applied; the altered genes included the oncogenes H-ras, c-fos and genes of HIV. Most of this work involved the modification of a target MRNA, based on a specific mutant codon that is cleaved by a specific ribozyme.

Several different ribozyme motifs have been described with RNA cleavage activity (Symons, 1992). Examples that are expected to function equivalently for the down regulation of mutant CD48, CD84, SLAM, 2B4, LY108, CS1, DEDD, NIT1, LY9, USF1 and/or GOLGA4 include sequences from the Group I self splicing introns including Tobacco Ringspot Virus (Prody et al, 1986), Avocado Sunblotch Viroid (Palukaitis et al., 1979; Symons, 1981), and Lucerne Transient Streak Virus (Forster and Symons, 1987). Sequences from these and related viruses are referred to as hammerhead ribozyme based on a predicted folded secondary structure.

Other suitable ribozymes include sequences from RNase P with RNA cleavage activity (Yuan et al., 1992, Yuan and Altman, 1994, U.S. Pat. Nos. 5,168,053 and 5,624,824), hairpin ribozyme structures (Berzal-Herranz et al, 1992; Chowrira et al., 1993) and Hepatitis Delta virus based ribozymes (U.S. Pat. No. 5,625,047). The general design and optimization of ribozyme directed RNA cleavage activity has been discussed in detail (Haseloff and Gerlach, 1988, Symons, 1992, Chowrira et al., 1994; Thompson et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A,C or U) (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in Cd48, Cd84, Slam, 2B4, LylO8, Cs1, Dedd, Nit1, Ly9, Usf1 and/or Golga4 targeted ribozymes is simply a matter of preparing and testing a given sequence, and is a routinely practiced “screening” method known to those of skill in the art.

5. Homologous Recombination

Although genetic transformation tends to be quite efficient, it is also accompanied by problems associated with random insertion. Random integration can lead to the inactivation of essential genes, or to the aberrant expression of the introduced gene. Additional problems associated with genetic transformation include mosaicism due to multiple integrations, and technical difficulties associated with generation of replication defective recombinant viral vectors.

Some of these drawbacks can be overcome by the utilization of a technique known as homologous recombination. This technique allows the precise modification of existing genes, overcomes the problems of positional effects and insertional inactivation, and allows the inactivation of specific genes, as well as the replacement of one gene for another. Methods for homologous recombination are described in U. S. Pat. No. 5,614,396, incorporated herein in its entirety by reference.

Thus a preferred method for the delivery of transgenic constructs involves the use of homologous recombination. Homologous recombination relies, like antisense, on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination is used as follows. First, a site for integration is selected within the host cell. Sequences homologous to the integration site are then included in a genetic construct, flanking the selected gene to be integrated into the genome. Flanking, in this context, simply means that target homologous sequences are located both upstream (5′) and downstream (3′) of the selected gene. These sequences should correspond to some sequences upstream and downstream of the target gene. The construct is then introduced into the cell, thus permitting recombination between the cellular sequences and the construct.

As a practical matter, the genetic construct will normally act as far more than a vehicle to insert the gene into the genome. For example, it is important to be able to select for recombinants and, therefore, it is common to include within the construct a selectable marker gene. This gene permits selection of cells that have integrated the construct into their genomic DNA by conferring resistance to various biostatic and biocidal drugs. In addition, this technique may be used to “knock-out” (delete) or interrupt a particular gene. Thus, another approach for inhibiting mutant CD48, CD84, SLAM, 2B4, LY108, CS1, DEDD, NIT1, LY9, USF1 and/or GOLGA4 involves the use of homologous recombination, or “knock-out technology”. This is accomplished by including a mutated or vastly deleted form of the heterologous gene between the flanking regions within the construct. The arrangement of a construct to effect homologous recombination might be as follows:

. . . vector•5′-flanking sequence•selected gene•selectable marker gene•flanking sequence-3′•vector . . .

Thus, using this kind of construct, it is possible, in a single recombinatorial event, to (i) “knock out” an endogenous gene, (ii) provide a selectable marker for identifying such an event and (iii) introduce a transgene for expression.

Another refinement of the homologous recombination approach involves the use of a “negative” selectable marker. One example of the use of the cytosine deaminase gene in a negative selection method is described in U.S. Pat. No. 5,624,830. The negative selection marker, unlike the selectable marker, causes death of cells which express the marker. Thus, it is used to identify undesirable recombination events. When seeking to select homologous recombinants using a selectable marker, it is difficult in the initial screening step to identify proper homologous recombinants from recombinants generated from random, non-sequence specific events. These recombinants also may contain the selectable marker gene and may express the heterologous protein of interest, but will, in all likelihood, not have the desired phenotype. By attaching a negative selectable marker to the construct, but outside of the flanking regions, one can select against many random recombination events that will incorporate the negative selectable marker. Homologous recombination should not introduce the negative selectable marker, as it is outside of the flanking sequences.

6. Marker Genes

In certain aspects of the present invention, specific cells are tagged with specific genetic markers to provide information about the fate of the tagged cells. Therefore, the present invention also provides recombinant candidate screening and selection methods which are based upon whole cell assays and which, preferably, employ a reporter gene that confers on its recombinant hosts a readily detectable phenotype that emerges only under conditions where a general DNA promoter positioned upstream of the reporter gene is functional. Generally, reporter genes encode a polypeptide (marker protein) not otherwise produced by the host cell which is detectable by analysis of the cell culture, e.g., by fluorometric, radioisotopic or spectrophotometric analysis of the cell culture.

In other aspects of the present invention, a genetic marker is provided which is detectable by standard genetic analysis techniques, such as DNA amplification by PCR™ or hybridization using fluorometric, radioisotopic or spectrophotometric probes.

a. Screening

Exemplary enzymes include esterases, phosphatases, proteases (tissue plasminogen activator or urokinase) and other enzymes capable of being detected by their activity, as will be known to those skilled in the art. Contemplated for use in the present invention is green fluorescent protein (GFP) as a marker for transgene expression (Chalfie et al., 1994). The use of GFP does not need exogenously added substrates, only irradiation by near UV or blue light, and thus has significant potential for use in monitoring gene expression in living cells.

Other particular examples are the enzyme chloramphenicol acetyltransferase (CAT) which may be employed with a radiolabelled substrate, firefly and bacterial luciferase, and the bacterial enzymes β-galactosidase and β-glucuronidase. Other marker genes within this class are well known to those of skill in the art, and are suitable for use in the present invention.

b. Selection

Another class of reporter genes which confer detectable characteristics on a host cell are those which encode polypeptides, generally enzymes, which render their transformants resistant against toxins. Examples of this class of reporter genes are the neo gene (Colberre-Garapin et al., 1981) which protects host cells against toxic levels of the antibiotic G418, the gene conferring streptomycin resistance (U. S. Pat. No. 4,430,434), the gene conferring hygromycin B resistance (Santerre et al., 1984; U. S. Pat. Nos. 4,727,028, 4,960,704 and 4,559,302), a gene encoding dihydrofolate reductase, which confers resistance to methotrexate (Alt et al., 1978), the enzyme HPRT, along with many others well known in the art (Kaufman, 1990).

D. Pharmaceutical Compositions

1. Pharmaceutically Acceptable Carriers

Aqueous compositions of the present invention comprise an effective amount of the CD48, CD84, SLAM, 2B4, LY108, CS1, DEDD, NIT1, LY9, USF1 and/or GOLGA4 protein, peptide, epitopic core region, inhibitor, nucleic acid sequence or such like, dissolved or dispersed in a pharmaceutically acceptable carrier or aqueous medium. Aqueous compositions of gene therapy vectors expressing any of the foregoing are also contemplated. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains a CD48, CD84, SLAM, 2B4, LY108, CS1, DEDD, NIT1, LY9, USF1 and/or GOLGA4 agent as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fingi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A CD48, CD84, SLAM, 2B4, LY108, CS1, DEDD, NIT1, LY9, USF1 and/or GOLGA4 protein, peptide, agonist or antagonist of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The active CD48, CD84, SLAM, 2B4, LY108, CS1, DEDD, NIT1, LY9, USF1 and/or GOLGA4 protein-derived peptides or agents may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

2. Liposomes and Nanocapsules

In certain embodiments, the use of liposomes and/or nanoparticles is contemplated for the introduction of CD48, CD84, SLAM, 2B4, LY108, CS1, DEDD, NIT1, LY9, USF1 and/or GOLGA4 protein, peptides or agents, or gene therapy vectors, including both wild-type and antisense vectors, into host cells. The formation and use of liposomes is generally known to those of skill in the art, and is also described below.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs). MLVs generally have diameters of from 25 mn to 4 μm. Sonication of MLVs results in the formation of small unilamellar vesicles (SUVs) with diameters in the range of 200 to 500 Å, containing an aqueous solution in the core.

The following information may also be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

E. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

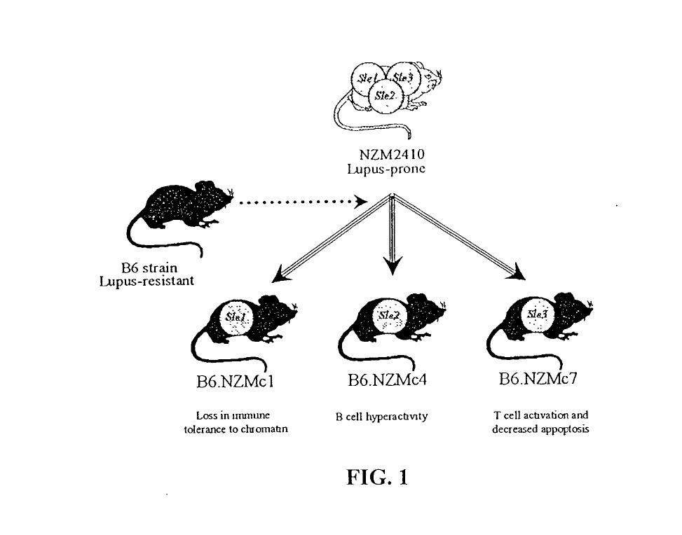

Congenic Dissection. Congenic dissection is a strategy in which each gene contributing to a polygenic disease (such as lupus) is segregated into an individual sub-strain of an inbred mouse strain. An example of this process is shown in FIG. 1, which presents results for the congenic dissection of the lupus-prone NZM2410 by introgression of three susceptibility intervals onto the non-autoimmune B6 inbred strain . This process created a collection of B6-congenic strains, each carrying a specific lupus-susceptibility gene in a genomic interval derived from NZM2410. Analyses of the immunologic phenotypes expressed by each congenic strain can then be used to identify the specific component phenotype that each contributes to the development of fatal autoimmune lupus nephritis in NZM2410 (Morel et al., 1996; Wakeland et al., 1997; Morel et al., 1997; Mohan et al., 1998; Morel et al., 1999; Morel et al., 1999).

Data indicates that three separate genetic pathways interact during the development of severe lupus nephlitis in this mouse model. During the first stage, genes such as Sle1 trigger the loss of immune tolerance to nuclear autoantigens and mediate the initiation of autoimmunity (Morel et al., 1996; Mohan et al., 1998). Genes in this pathway are capable of causing the initiation of a humoral autoimmune response to nuclear antigens; however, this response is not pathogenic in the absence of genes in the other pathways. In this regard, many first-degree relatives of SLE probands exhibit a similar seropositive phenotype without severe disease pathogenesis (Winchester, 1992). The second genetic pathway mediating lupus susceptibility contains genes causing generalized immune hyper-responsiveness or dysregulation. Genes such as Sle2 (Mohan et al., 199X), Sle3(Mohan et al., 199X), lpr (Cohen et al., 1991), gld (Cohen et al., 1991), and Yaa (Izui et al., 1994) would all be included in this pathway. These genes often do not generate autoimmune phenotypes in lupus-resistant genomes, but strongly enhance the expansion of the autoimmune response when combined with genes that mediate the loss of tolerance to nuclear autoantigens (Mohan et al., 1997). The final class of lupus susceptibility genes are those that potentiate end organ damage. Theoretically, end organ damage could be enhanced by a variety of molecular mechanisms, including genes that modify immune effector functions (such as Fc receptors (Clynes et al., 1998)) and those that modify the end organ itself.

Based upon an analysis of the epistatic interactions that occur when SLE susceptibility genes are re-combined on the B6 background, fatal lupus nephritis is produced when Sle1 is combined with either Sle2 or Sle3 on the non-autoimmune B6 genome (Mohan et al., 1999). This is the first example of the successful reconstruction of a complex disease phenotype on a normal genome via the reassembly of congenic intervals. A key aspect of this finding is the observation that two genes in combination are sufficient to generate fatal lupus nephritis. This observation makes feasible the unambiguous identification of the disease genes in each of these intervals via assaying their capacity to elicit fatal lupus when combined with the genes in a second congenic strain. For example, the gene(s) in the Sle1 interval that is essential for disease will produce fatal lupus when they are bred with B6.NZMc7 (which carries the intact Sle3 interval).

Congenic strain construction is a requisite initial stage in identifying the genes causing murine lupus. Each congenic strain becomes a monogenic model for a specific component phenotype associated with the disease process. The inventors are mapping the positions of each of the Sle genes using this approach with analysis of Sle1 is most advanced. The inventors have characterized over 2000 testcross progeny and created a collection of congenic recombinant strains with truncated segments of the B6.NZMc1 interval. A detailed analysis of the phenotypes expressed by these recombinants reveals several important findings about Sle1. First, Sle1 is not a single gene, but is instead a gene cluster containing at least 4 genes that impact susceptibility to autoimmune lupus nephritis. All four genes, which have been designated Sle1a-Sle1d, have been isolated on truncated congenic sub-intervals. Phenotypic analyses of these subintervals indicate that each gene in the Sle1 cluster expresses a unique subset of the multiple component phenotypes associated with the Sle1 interval.

Two features of the genes in the Sle1 cluster suggest that they may have closely-related functions. First, both Sle1a and Sle1b break tolerance to nuclear chromatin in a manner resulting in the preferential production of IgG autoantibodies against H2A/H2B-DNA subnucleosome antigens. A second intriguing feature of this system is our recent observation that Sles1, a locus-specific suppressive modifier located on chromosome 17 in the NZW genome, can suppress all the autoimmune phenotypes produced by the entire Sle1 cluster (Morel et al., 1999). The ability of Sles1 to suppress all of the component phenotypes associated uniquely with Sle1a, Sle1b, and Sle1d suggests that all these genes function within a single biologic pathway that mediates a breach in tolerance to chromatin autoantigens. Clearly, identifying the genes in this cluster will provide crucial insights into the manner in which immune tolerance to nuclear antigens is broken, as well as the fashion in which the potentially benign autoantibody phenotype associated with Sle1 in isolation becomes pathogenic in the presence of other susceptibility genes.

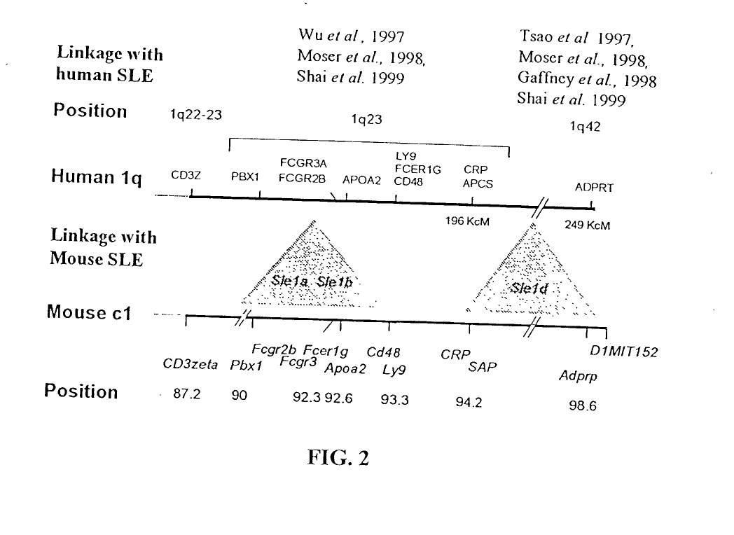

The results of several recent linkage studies of SLE in humans reveal an intriguing relationship between the genetics of susceptibility in humans and mice. The region of the human genome that is syntenic with the interval containing the murine Sle1 gene contains a cluster of genes associated with human SLE susceptibility (Tsao et al., 1997; Harley et al., 1998; Moser et al., 1998; Gafffney et al., 1998; Shai et al., 1999). The precise positions within this interval that are most strongly associated with human SLE susceptibility have varied somewhat in the findings of different groups, and in some cases multiple peaks have been detected in the 1q23 to 1q42 region. As shown in FIG. 2, the Sle1 gene cluster is located within a region of murine chromosome 1 that is syntenic with the 1q23 to 1q42 region in humans, strongly supporting the contention that the human homologue of the Sle1 gene cluster is a key genetic predisposing factor for human SLE. In this regard, the distance between CRP and ADPRT in humans is much longer (˜55 cM) than the distance from Crp to Adprp in mouse (˜8 cM), suggesting that synteny within this region may not be complete. Nonetheless, this region remains a strong example of homologous mapping of disease susceptibility genes in humans and mice.

### EXAMPLE 2

Genetic and phenotypic properties of genes in the Sle1 cluster. The initial characterization of the phenotypic properties of B6.NZMc1 indicated that this interval contained a gene or genes that mediated the spontaneous production of IgG autoantibodies directed against nuclear chromatin (Morel et al., 1997). Subsequently, more detailed analyses revealed a variety of additional component phenotypes associated with this interval, most notably: (1) the predominant production of autoantibodies detecting H2A/H2B-DNA subnucleosomes (Mohan et al., 1998); (2) hypergammaglobulinemia (Mohan et al., 1998); (3) expanded splenicCD4+CD69+ activated T cell population (Mohan et al., 1998); (4) increased expression of B7.2 on splenic B cells (Sobel et al., 1999); and (5) enhanced immune responsiveness. Although these phenotypes have not been completely characterized, in heterozygotes, the penetrance of autoantibody phenotypes in heterozygotes (B6×B6.NZMc1 hybrids carrying one NZM2410-derived Sle1² allele and one B6-derived Sle1^{b} allele) is significantly lower than that of homozygous B6.NZMc1 mice (Morel et al., 1997). This allele-dose mode of inheritance is consistent with the properties of Sle1 from an original genome scan and indicated that phenotypic expression of Sle1 is detectable in a heterozygote, but is much more strongly expressed in homozygotes.

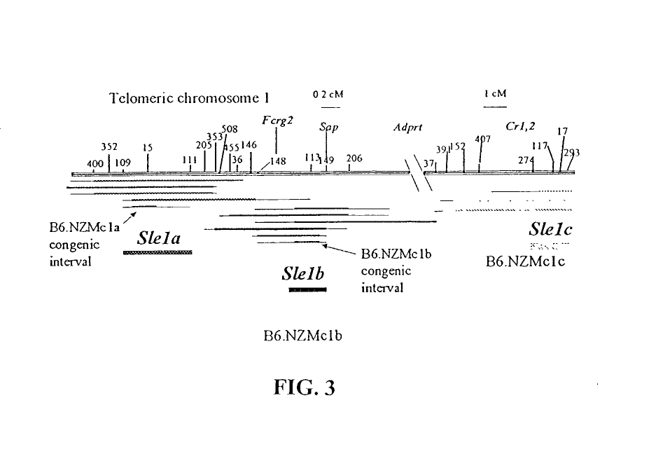

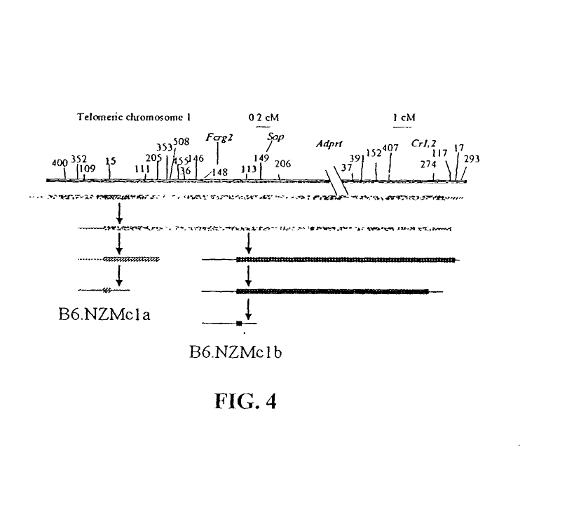

The approach adopted to mapping these traits within the Sle1 interval was to create an extensive collection of recombinant chromosomes throughout the interval and to assess these phenotypes in individual congenic recombinant strains. Over 2400 testcross progeny were subsequently generated, originally with the intact B6.NZMc1 interval and subsequently with several truncated sublines, and characterized the over 500 recombinational breakpoints created within the Sle1 congenic interval. All recombinant progeny carrying potentially useful recombinant chromosomes were subsequently bred and their progeny were aged and tested for autoantibody production. This analysis ultimately led to the creation of a large collection of congenic recombinants with truncated congenic intervals throughout the Sle1 region, as well as to the realization that more than one gene capable of breaking tolerance to nuclear chromatin resided within the Sle1 interval. Some of the truncated intervals that led to the delineation of three of these loci are presented in FIG. 3.

A summary of the immunologic phenotypes expressed by the congenic recombinant strains defining the first three loci detected in the Sle1 interval are presented in Table 1. These results illustrate the remarkable overlap in phenotype between the genes within the Sle1 cluster. However, each gene has at least one feature that is distinct from that of all the other genes in the cluster. These findings are consistent with the notion that all these genes are part of a functionally related cluster that in some fashion can impact the maintenance of immunologic tolerance to nuclear antigens.

TABLE 1
Component phenotypes associated with each of the three susceptibility
genes detected in the B6.NZMc1 congenic interval

| Phenotype | Sle1a | Sle1b | Sle1c |
|---|---|---|---|
| Anti-chromatin IgG | + | ++ | + |
| H2A/H2B/DNA specificity | + | + | − |
| B cell activation | − | + | − |
| CD4 T cell activation | + | + | − |
| Heightened IgG immune response | + | − | − |
| T cell % | ↓ | ←→ | ←→ |

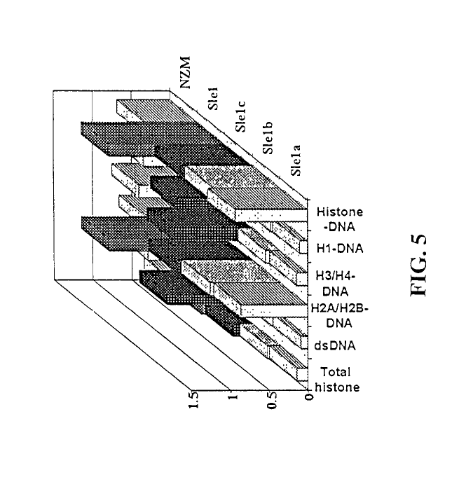

Fine mapping Sle1a and Sle1b. Sle1a and Sle1b were originally localized to a 2.4 cM interval (B6.NZMc1₄₀₀₋₂₀₆) containing D1MIT15, the peak marker locus for Sle1 in the inventors original genome scan. Subsequently, Sle1a and Sle1b were localized to separate congenic intervals that were sequentially shortened via the analysis of new recombinants. The series of recombinant chromosomes that led to the derivation of the B6.NZMc1a and B6.NZMc1b congenic strains are presented in FIG. 4. The congenic intervals defining the locations of Sle1a and Sle1b are smaller than 0.4 cM in size, each carrying an NZM2410-derived allele for only a single polymorphic microsatellite marker that distinguishes B6 from NZM2410. B6.NZMc1a carries an NZM2410 marker for D1MIT15 and is B6-derived for D1MIT109 and D1MIT111, while B6.NZMc1b carries an NZM2410 marker for D1MIT113 and is B6-derived at DlMIT148 and D1MIT149. These two sub-intervals are extremely small and represent segments that are <0.4 cM in length. The precise borders of these intervals have been further refined as the inventors have developed physical maps across these regions.

Phenotypic analyses of the minimal intervals defining Sle1a and Sle1b led to the determination that IgG anti-chromatin autoantibodies are produced with high penetrance and continue to have an allele dose/recessive mode of expression with all three genes. The specificity of these autoantibodies with respect to their recognition of subnucleosome components has also been characterized and the results of this analysis presented in FIG. 5. Both Sle1a and Sle1b mediate the preferential recognition of the H2A/H2B-DNA subnucleosome component of nuclear chromatin, similar to the results obtained with the entire Sle1 interval. Sle1c, in contrast, does not show this specificity and mediates the production of relatively low titered autoantibodies against all of the subnucleosome components of chromatin. These results support the hypothesis that Sle1a and Sle1b may be two genes within a single, biological pathway that, when disrupted in some fashion, mediates the loss of tolerance to chromatin via a specific mechanism.

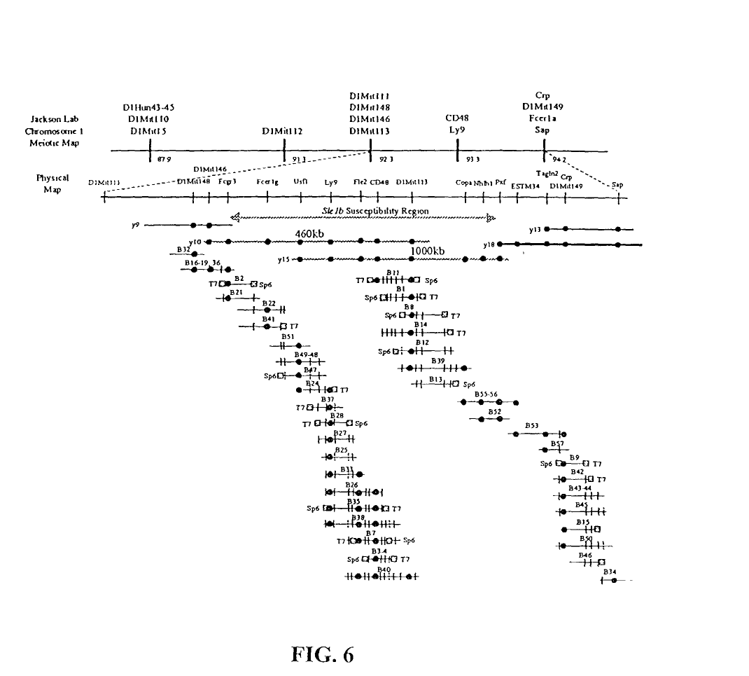

Development of physical maps across Sle1a and Sle1b critical intervals. The truncated congenic strains defined in FIG. 4 each carry extremely small genomic intervals derived from the lupus-prone NZM2410 strain and strongly express several key intermediate autoimmune phenotypes. The NZM2410-derived congenic intervals in these strains unambiguously define the critical regions for Sle1a and Sle1b in the genome and thus facilitate the development of physical maps across these regions. Results of the production of a YAC and BAC contig across Sle1b are presented in FIG. 6.

The YAC contig spanning Sle1b was produced by screening the MIT YAC library (produced from the C57BL/6 genome) available through Research Genetics. This analysis determined that the critical interval for Sle1b is spanned by two overlapping YAC clones (shown in tan), leading to an estimate of <1.4 million basepairs for the Sle1b critical interval, based on CHEF gel sizing of these YACs. In order to develop the resources required to identify Sle1b required the development of a sequence-ready BAC contig across the Sle1b interval. To produce this contig, the C57BL/6-derived BAC library produced by DeJong and co-workers (Osoegawa et al., 1998) was screened using traditional positional cloning strategies. This evaluation eventually produced a contig containing a total of 57 BACs ordered with 75 markers and STSs which spanned the entire Sle1b interval. Since it has been estimated that 1-5% of all BAC clones are chimeric (McPherson et al., 1997), several confirmation strategies must be employed to verify the integrity of a BAC contig prior to the initiation of large scale DNA sequencing. Sle1b region BACs were thus analyzed by restriction digest fingerprinting and FISH (to confirm hybridization to chromosome 1) (McPherson et al., 1997; Vollrath et al., 1999; Wandstrat et al., 1998). It was confirmed that the BAC contig spanned the Sle1b interval and the positions of the ends of the congenic intervals were localized with resolution of ˜50 Kb.

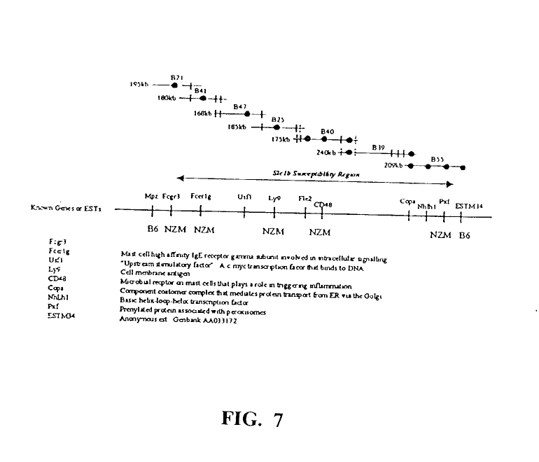

The critical interval for Sle1b is spanned by a “tiling path” of seven BACs, indicating that Sle1b has been localized into a region containing ˜1.0 Mbases of genomic DNA. These “tiling path” BACs, together with a physical map of the critical region for Sle1b, are presented in FIG. 7. Based on database searches with BAC end sequences and from pre-existing YAC contigs in this region (Underhill et al., 1999; Eddleston et al., 1999) more than 25 genes or ESTs were identified within this critical region. The primary candidate genes are included in the physical map, each localized to a specific BAC within the contig. At this point, the four best candidate genes within this region are Fc receptor III alpha chain (expressed predominantly in neutrophils and NK cells), Fc epsilon receptor 1 alpha (expressed on mast cells and basophils), CD48 ( CD2 ligand, expressed in B cells) and Ly9 (classic cell surface antigen on lymphocytes).

Polymorphisms in the FcRIII gene in humans have been associated with SLE susceptibility in several studies (Salmon et al., 1996; Gibson et al., 1999; Wu et al., 1997a & 1997b), thus making the characterization of the murine homologue of this gene contig a top priority. However, some functional properties of this receptor make it less attractive as a candidate for the component phenotypes associated with Sle1b. First, the in vivo phenotypes impacted by FcRIII have been characterized in detail, and a variety of studies clearly indicate that FcRIII is not expressed in B cells and that it functions predominantly or even exclusively in effector mechanisms such as ADCC and the induction of antibody-mediated inflammatory reactions (Ravetch et al., 1998). Further, the targeted disruption of FcRIll has been shown to have no effect on humoral immune responses or the development of autoantibodies in the NZB/W model of SLE (Clynes et al., 1998). Sle1b, on the other hand, is expressed in B cells and mediates the loss of tolerance to chromatin antigens and the production of autoantibodies. Thus, the known functions of FcRIII do not readily account for the component phenotypes associated with Sle1b. Nonetheless, we cannot exclude the possibility that the Sle1b-associated phenotypes reflect unrecognized functions of FcRIII. Consequently, the first BAC insert that will be analyzed in detail will be BAC21 which overlaps with the end of the congenic interval in B6.NZMc1b and contains FcRIII.

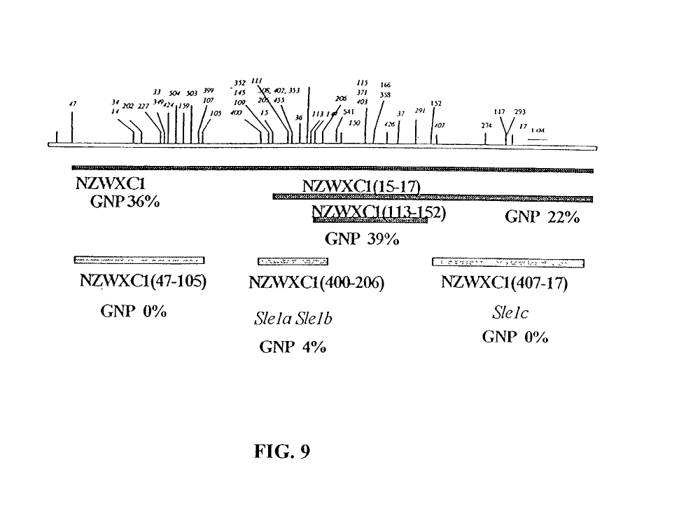

A C57BL/6-derived BAC contig across the Sle1a critical interval was developed. The current status of this project is presented in FIG. 9. A C57B1/6-derived contig across Sle1a by screening the De Jong library in a similar fashion is used to produce the contig across Sle1b.

Sequence analysis of BAC 21 in the Sle1b contig. In order to perform sequence analysis of the Sle1b contig with BAC 21, which contains both FcRIll and the centromeric border of the B6.NZMc1b congenic interval, sheared DNA libraries were produced from individual BAC clones and ordered shotgun sequencing performed to produce a sequence coverage of >5 fold redundancy. Thus, to determine the sequence of BAC21, which is approximately 195 kb in length (see FIG. 7), more than 2000 sequence reads of >500 bp are required. DNA sequencing is performed using a Beckman CEQ 2000 capillary sequencer, which is fully automated and capable of producing >670 sequences/week (projected average read length of greater than 600 bp). Sequence assembly is performed using software supplied with the Beckman CEQ 2000 and Sequencher (Gene Codes Corp, Michigan).

The key element in this process is the production of high quality libraries of sheared DNA fragments from the BAC. To produce these libraries, isolated large quantities of individual BAC DNAs were isolated following the protocol from the Wellcome Trust Centre for Human Genetics and Institute of Molecular Medicine, Oxford. Briefly, 500-600 μg of BAC DNA is recovered by alkaline lysis from 1 liter of BAC cells grown overnight. Pellets were lysed and purified using RNase, proteinase K, phenol-chloroform extractions, and ethanol precipitation. DNA was then quantitated using a fluorometer and assessed for quality via digested with NotI and electrophoretic analysis on a CHEF gel.

To make the sheared libraries, approximately 50 ,μg of BAC DNA is diluted to 25 μg/ml in TE and run through a nebulizer (Glas-Col, Inc.; Terre Haute, Ind.) at 150 psi nitrogen for 2.5 minutes. A nebulizer was chosen to shear the BAC DNA as sonication or other shearing methods can cause DNA tearing and reduce the amount of clonable DNA. Shearing efficiency is very high using this method with the majority of the DNA in the 300-1000 bp range. The ends of 25 μg DNA is then mended with 0.2 mM dNTPs, 40 units T4 DNA polymerase. This reaction is incubated for 15 min. at room temperature and then 25 units Klenow is added and the whole reaction incubated another hour at RT. This DNA is then purified, run on a 1% agarose gel, and fragments in the 300-1000 bp size range are excised, purified using the Genecleanll kit (BIO101), and quantitated using a fluorimeter. Inserts are then phosphorylated and purified for ligation into the pUK18 vector. pUK18 vector is prepared for ligation by digestion with Smal, followed by purification on an agarose gel, and treatment with shrimp alkaline phosphatase. Ligations are set up in 20 ul reactions using the Rapid Ligation Kit (Roche Boehringer Mannheim), transformed into DH5α™ competent cells (GibcoBRL), and plated onto LB plates containing 100 μg/ml ampicillin, 50 μg/ml X-gal, and lmM IPTG for blue-white selection.

To assess the quality of the library, approximately 24 colonies are picked, grown in 3-5 ml LB/amp, and screened for inserts. If this analysis yields satisfactory efficiency and insert size, ligation reactions are scaled up and approximately 20-30 96-well plates are picked (1800-2880 clones), resulting in a library of clones sufficient for a 6-9 fold coverage of the 195 kb BAC 21. The Qiagen Turbo kit for 96-well DNA minipreps will be used to purify the DNA for sequencing.

Detection of Sle1d. Identifying the gene or combination of genes within this cluster that is essential for the production of fatal disease is an important issue raised by the detection of multiple susceptibility loci within the Sle1 interval. An initial analysis of this issue was carried out by measuring lupus in F1 hybrids between B6.NZMcl sub-congenic intervals and NZW. This strategy was developed based on the recent demonstration that (B6.NZMc1×NZW)F1 hybrids developed fatal lupus, while (B6×NZW)F1 hybrids were healthy (Morel et al., 1999b). The genetic basis for the development of fatal lupus in (B6.NZMc1×NZW)F1 hybrids is complex, resulting from an epistatic interaction of homozygous Sle1 with Sle3 and Sle6 and the simultaneous inactivation of four suppressive modifiers. The key aspect relevant to an analysis of the genes within the Sle1 cluster that are required for the development of fatal disease is the dramatic variation in phenotype between (B6×NZW)F1 hybrids (healthy) and (B6.NZMc1×NZW) F1 hybrids (fatal lupus). To identify which portion(s) of the Sle1 gene cluster are required for disease development, cohorts of F1 hybrids were produced between individual sub-congenic intervals of the Sle1 cluster and NZW and aged them through 12 months to assess their susceptibility to fatal lupus nephritis.

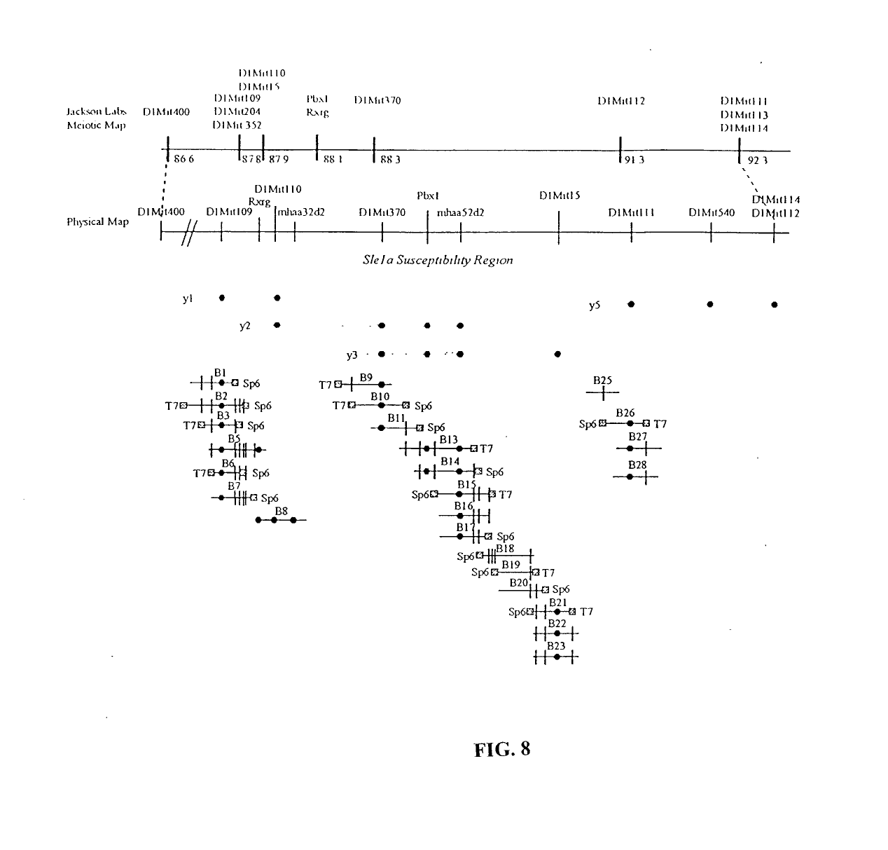

As shown in FIG. 9, this analysis revealed several interesting features of the fashion in which this gene cluster interacts with other lupus susceptibility genes to cause severe disease. First, Sle1c is incapable of interacting with Sle3 and Sle6 to cause disease. Similarly, regions centromeric to Sle1a did not result in detectable disease. In this regard, the region centromeric to D1MIT400 in the congenic interval of B6.NZMc1 has not expressed any autoimmune component phenotypes throughout the study, thus excluding this region as a site of additional susceptibility genes. Interestingly, Sle1a and Sle1b in combination (designated NZW×Cl(400-206) in FIG. 8) only generate very minimal disease (4% fatal lupus). These results indicate that neither of these genes two genes in association are insufficient to regenerate the severe epistatic disease mediated by the full Sle1 interval. The strongest effect was clearly localized to a ˜9 cM interval beginning with Sle1b and extending telomeric to marker D1MIT152. This segment generated fatal lupus with a penetrance similar to that obtained for the entire Sle1 gene cluster (36% for NZW×C1 versus 39% for NZW×C1(113-152). These results indicate that an additional susceptibility gene, which has been designated Sle1d, is located within a 9 cM interval telomeric to Sle1b. Whether Sle1d is capable of interacting with Sle3 and Sle6 to cause fatal lupus in the absence of Sle1b is an additional issue that remains to be resolved.

Genetic dissection of Sle3 and Sle5 in B6.NZMc7. An initial analysis of B6.NZMc7 indicated that the susceptibility gene in this interval caused a dysregulation of the immune system leading to polyclonal/polyreactive IgG antibody production against a variety of self and non-self antigens, resulting in the production of low-titered autoantibody against nuclear antigens and causing significant levels of glomerulonephritis (Morel et al., 1997). Subsequently, more detailed analyses revealed a variety of additional component phenotypes associated with this interval, most notably: (1) lowered threshold of activation for CD4+ T cells; (2) increased splenic CD4:CD8 ratio; (3) increased CD4+CD69+ activated T cells in spleen; (4) lowered rate of activation-induced cell death in CD4+ splenic cells; and (5) disorganized splenic T cell and B cell zones (Mohan et al., 1999). Finally, studies indicate that the expansion of the CD4 compartment occurs in both B6- and B6.NZMc7-derived T cells in mixed-bone marrow chimeras. These observations suggest that the immune dysfunction expressed in B6.NZMc7 may not be intrinsic to T lymphocytes, but rather caused by an antigen-presenting cell, possibly in the monocyte/dendritic cell lineage.

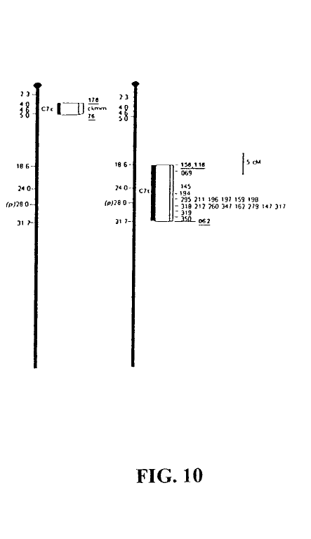

Initial genome scans in crosses between B6 and NZM2410 suggested that two separate susceptibility genes might be present in the centromeric segment of chromosome 7 (Morel et al., 1994; Morel et al., 1999). This was subsequently confirmed by the production of two sub-lines, designated B6.NZMc7c and B6.NZMc7t, each of which mediated the expression of some elements of the autoimmune phenotype expressed by B6.NZMc7. The locations of the truncated congenic intervals in these two strains are presented in FIG. 10. Analysis indicates that B6.NZMc7t expresses most of the key T lymphocyte phenotypes.

TABLE 2
CD4:CD8 ratio and activated CD4 population is only expanded in
B6.NZMc7t

| Cell | Spleen cells ex vivo from | | |
|---|---|---|---|
| Population | B6 | B6.NZMc7c | B6.NZMc7t |
| % CD4 T cells | 17.45 ± 2.97^{a} | 17.42 ± 2.43 | 20.22 ± 4.23*^{b} |
| % CD8 T cells | 11.73 ± 2.48 | 11.78 ± 2.11 | 10.39 ± 2.53 |
| CD4:CD8 T cell ratio | 1.51 ± 0.16 | 1.49 ± 0.15 | 2.01 ± 0.45*** |
| CD69⁺CD4 T cells | 16.4 ± 9.3 | 20.3 ± 9.3 | 25 ± 7** |
| N^{c} | 26 | 15 | 17 |

B6.NZMc7t develops hypergammaglobulinemia, autoantibodies, and glomerulonephritis, consistent with the presence of a gene mediating the majority of the phenotypes associated with Sle3. Consequently, the gene in B6.NZMc7t has been designated as Sle3. B6.NZMc7c also mediates the production of autoantibodies and lower levels of glomerulonephritis. This gene has been designated Sle5.

Phenotypic analysis with gene expression microarrays. Gene expression microarray analysis using the CLONTECH membrane system has been developed in order to provide insights into the genetic pathways that each susceptibility interval dysregulates and to provide important insights into the molecular mechanisms that are impaired by individual susceptibility genes. It is anticipated that this mode of analysis may ultimately allow a detailed molecular analysis of genetic pathways influenced by individual susceptibility genes and specific combinations of susceptibility genes. In addition, variations in gene expression can be analyzed genetically as a phenotype mediated by a gene or genes within the congenic interval. Theoretically, variations in gene expression patterns detected with microarrays may be useful as intermediate phenotypes for the detection of more complex (and potentially less penetrant) phenotypes. For example, it is anticipated that gene expression patterns in B6.NZMc7t CD4+ T cells will vary significantly from B6 as a consequence of the influences of Sle3 on T cell activation and CD4 expansion, even though Sle3 may not be intrinsically expressed in T cells. Nonetheless, if Sle3 in the B6.NZMc7t congenic interval mediates changes in CD4 T cell gene expression patterns, these changes can be used to identify the location of Sle3 among congenic recombinants. Thus, gene expression microarray analysis can be coupled with congenic recombinant analysis to identify epistatic genetic modifiers influencing specific genetic pathways in lymphocytes.

The value of this technology is dependent upon the reproducibility and accuracy of the expression array phenotypes. Consequently, an initial focus was on characterizing variations in gene expression patterns of T and B cells isolated from the NZM2410 and B6 parental strains. These cells were isolated using magnetic bead technologies. Specifically, CD4 T cells are isolated from spleen cells depleted of red blood cells via incubation with anti-mouse CD4 mAb-coupled to magnetic beads. After isolation of the CD4 T cells, the remaining cells are incubated with a mixture of biotinylated mAbs specific for macrophages (Mac-1), granulocytes (Gr-1), CD8 T cells (CD8) and NK cells (NKI.1). After removal of these cells with streptavidin-coupled magnetic beads, the negatively selected B cell population is recovered. FACS analysis of these purified populations indicate that the positively-selected CD4+ T cell population is more highly purified (˜95% CD4+) than the negatively selected B cells (˜85% B220+). From one mouse spleen, 5-10 million CD4 T cells and 40-60 million B cells can be obtained. Consequently, 3 spleens/experiment are necessary to obtain sufficient cells for the preparation of cDNA probes.

Total cellular RNA is prepared from each isolated cell population, treated with DNAseI to remove genomic DNA, and used as template for the generation of ³²P labeled cDNA probes by reverse transcription. The probes are hybridized to the cDNA expression arrays which include 588 cDNAs spotted in duplicate on a positively charged nylon membrane. Quantitative analysis of hybridization is obtained using a Molecular Dynamics phosphorimager and the images are analyzed with Atlasimage software from CLONTECH. Side-by-side hybridizations with complex cDNA probes prepared from two different RNA sources allow the simultaneous comparison of expression levels for 588 cDNAs in the source material. The genes from which these cDNAs are generated have been reported to play key roles in many different biological processes and are characterized by tight transcriptional regulation. The cDNAs in this array allow an assessment of gene expression in several key cellular and immunologic functions (for details, see CLONTECH catalogue).

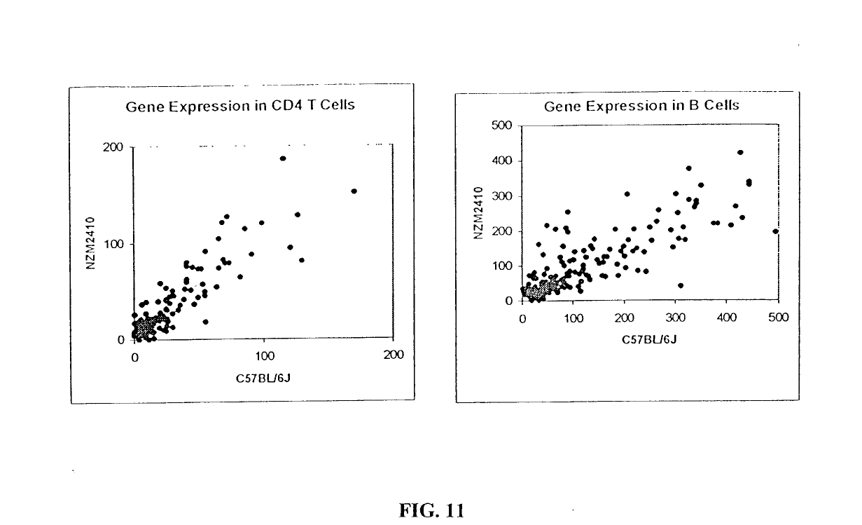

The results of an analysis of CD4+ T cells and B cells from B6 and NZM2410 is presented in FIG. 11. These data suggest that B6 and NZM2410 vary significantly in their gene expression patterns. A listing of all the genes with significant variations in their expression (approximately 2 fold difference or greater) between these two parental strains is presented in Table 3 on the following page. This list clearly indicates that this assay can provide evidence to support virtually any candidate gene hypothesized to impact autoimmunity. Genes effecting apoptosis, cell cycling, and a variety of interleukin receptors all showed significant variations in expression between B6 and NZM2410. Many of these variations undoubtedly reflect the hyperactive state of lymphocytes in the chronically autoimmune NZM2410. Nonetheless, these variations (if they are reproducible) support the hypothesis that this technology will identify valuable intermediate phenotypes for mapping studies of individual susceptibility genes. Interestingly, splenic B cells exhibited significantly greater variations in gene expression than T cells. This is consistent with previous studies which have indicated that B cells are more severely dysregulated than T cells in the NZB/NZW model (reviewed in Mohan et al., 1998).

TABLE 3
Summary of individual genes with variations in expression between
B6 and NZM2410

| | Adj.In- | Adj.In- | | |
|---|---|---|---|---|
| CD4 T Cells | tensity- | tensity- | | |
| Protein/gene | B6 | NZM2410 | | |
| B-myb proto-oncogene; myb-re- | 2 | 18 | Empty | 16 |
| lated protein B | | | | |
| casein kinase II (alpha subunit) | 22 | 10 | 0.454545 | −12 |
| H-ras proto-oncogene; trans- | 4 | 0 | Empty | −4 |
| forming C-protein | | | | |
| cyclin E (G1/S-specific) | 5 | 21 | 4.2 | 16 |
| extracellular signal-regulated | 9 | 27 | 3 | 18 |
| kinase 1 (ERK 1); p44; Ert2 | | | | |
| Rsk; ribosomal protein S6 kinase | 18 | 39 | 2.166667 | 21 |
| Zyxin; LIM domain protein; | 130 | 81 | 0.623077 | −49 |
| alpha-actinin binding protein | | | | |
| BAG-1; bcl-2 binding protein | 1 | 17 | Empty | 16 |
| with anti-cell death activity | | | | |
| Bak apoptosis regulator; Bcl-2 | 12 | 22 | 1.833333 | 10 |
| family member | | | | |
| Bax; Bcl2 heterodimerization | 6 | 14 | 2.33333 | 8 |
| partner and homolog | | | | |
| Bcl-2; B cell lymphoma protein | 4 | 7 | 1.75 | 3 |
| 2; apoptosis inhibitor | | | | |
| PD-1 prossible cell death indu- | 25 | 14 | 0.56 | −11 |
| cer; 1g gene superfamily | | | | |
| member | | | | |
| tumor necrosis factor receptor 1 | 55 | 18 | 0.327273 | −37 |
| (TNFR-1) | | | | |
| basic domain/leucine zipper | 9 | 2 | Empty | −7 |
| transcription factor | | | | |
| GATA-3 transcription factor | 3 | 18 | Empty | 15 |
| interleukin-2 receptor gamma | 6 | 36 | 6 | 30 |
| chain | | | | |
| Neuroleukin | 366 | 171 | 0.467213 | −195 |
| interleukin 6 (B cell | 9 | 16 | 1.777778 | 7 |
| differentiation factor) | | | | |
| B Cells | | | | |
| Protein/gene | Adj | Adj | | |
| p107; RBL1; retinoblastoma | 11 | 38 | Empty | 27 |
| gene product-related protein | | | | |
| p107 (cell cycle regulator) | | | | |
| Rb; pp105; retinoblastoma | 44 | 77 | 1.75 | 33 |
| susceptibility-associated protein | | | | |
| (tumor suppressor gene; cell | | | | |
| cycle regulator) | | | | |
| B-myb proto-oncogene; myb- | 67 | 205 | 3.059701 | 138 |
| related protein B | | | | |
| c-Jun proto-oncogene | 578 | 799 | 1.382353 | 221 |
| (transcription factor AP-1 | | | | |
| component) | | | | |
| RNA polymerase I termination | 1984 | 6102 | 3.075605 | 4118 |
| factor TTF-1 | | | | |
| c-rel proto-oncogene | 25 | 7 | Empty | −18 |
| Pim-proto-oncogene | 208 | 303 | 1.456731 | 95 |
| c-Fes proto-oncogene | 14 | 29 | 2.071429 | 15 |
| H-ras proto-oncogene; | 37 | 8 | Empty | −29 |
| transforming G-protein | | | | |
| cyclin A (G2/M-specific) | 24 | 80 | 3.333333 | 56 |
| cyclin B1 (G2/M-specific) | 28 | 64 | 2.285714 | 36 |
| cyclin B2 (G2/M-specific) | 42 | 133 | 3.166667 | 91 |
| cyclin D3 (G1/S-specific) | 6 | 21 | Empty | 15 |
| cyclin E (G1/S-specific) | 50 | 217 | 4.34 | 167 |
| Cdk4; cyclin-dependent kinase 4 | 76 | 125 | 1.644737 | 49 |
| Wee1/p87; cdc 2 tyrosine 15- | 19 | 32 | 1.684211 | 13 |
| kinase | | | | |
| 1-kB (1-kappa B) beta | 115 | 26 | 0.226087 | −89 |
| Stat1; signal transducer and | 494 | 195 | 0.394737 | −299 |
| activator of transcription | | | | |
| Stat3; acute phase response | 29 | 10 | Empty | −19 |
| factor (APRF) | | | | |
| Fyn proto-oncogene; Src family | 54 | 23 | 0.425926 | −31 |
| member | | | | |
| MAPKK1; MAP kinase kinase 3 | 88 | 43 | 0.488636 | −45 |
| (dual specificity) (MKK1) | | | | |
| PKC-beta; protein kinase C | 112 | 40 | 0.357143 | −72 |
| beta-II type | | | | |
| Bcl-2; B cell lymphoma protein | 40 | 20 | 0.5 | −20 |
| 2; apoptosis inhibitor | | | | |
| DAD-1; defender against cell | 188 | 71 | 0.37766 | −117 |
| death 1 | | | | |
| FLIP-L; apoptosis inhibitor; | 18 | 4 | Empty | −14 |
| FLICE-like inhibitory protein | | | | |
| gadd45; growth arrest and DNA- | 24 | 64 | 2.666667 | 40 |
| damage-inducible-protein | | | | |
| p55cdc; cell division control | 90 | 254 | 2.822222 | 164 |
| protein 20 | | | | |
| PD-1 possible cell death inducer, | 12 | 26 | 2.166667 | 14 |
| 1g gene superfamily member | | | | |
| tumor necrosis factor receptor 1 | 245 | 81 | 0.330612 | −164 |
| (TNFR-1) | | | | |
| DNA polymerase alpha catalytic | 2 | 36 | Empty | 34 |
| subunity (p180) | | | | |
| DNA topoisomerase II (Top II) | 4 | 28 | Empty | 24 |
| MHR23A; Rad23 UV excision | 81 | 157 | 1.938272 | 76 |
| repair protein homolog; | | | | |
| xeroderma pigmentosum | | | | |
| group C (SPC) repair | | | | |
| complementing protein | | | | |
| PCNA; proliferating cell nuclear | 13 | 50 | 3.846154 | 37 |
| antigen; processivity factor | | | | |
| early B cell factor (EBF) | 43 | 15 | 0.348837 | −28 |
| DNA-binding protein SATB 1 | 227 | 84 | 0.370044 | −143 |
| DP-1 (DRTF-polipeptide 1) cell | 50 | 92 | 1.84 | 42 |
| cycle regulatory transcription | | | | |
| factor | | | | |
| Elf-1 (Ets family transcription | 71 | 31 | 0.43662 | −40 |
| factor) | | | | |
| erythroid kruppel-like | 87 | 209 | 2.402299 | 122 |
| transcription factor | | | | |
| PAX-5 (B cell specific | 1052 | 426 | 0.404943 | −626 |
| transcription factor) | | | | |
| transcription factor RelB | 40 | 16 | 0.4 | −24 |
| Erthropoietin receptor | 33 | 163 | 4.909091 | 129 |
| interferon-gamma receptor | 84 | 37 | 0.440476 | −47 |
| interleukin-10 receptor | 33 | 3 | Empty | −30 |
| interleukin-2 receptor gamma | 898 | 344 | 0.383074 | −554 |
| chain | | | | |
| interleukin-4 receptor | 43 | 16 | 0.372093 | −27 |
| (membrane-bound form) | | | | |
| interleukin-9 receptor | 25 | 8 | Empty | −17 |
| insulin-like growth factor | 71 | 23 | 0.323944 | −48 |
| binding protein-4 (IGFBP-4) | | | | |
| mothers against DPP protein | 8 | 25 | Empty | 17 |
| (mad homolog Smad 1); | | | | |
| transforming growth factor | | | | |
| beta signaling protein | | | | |

### EXAMPLE 3

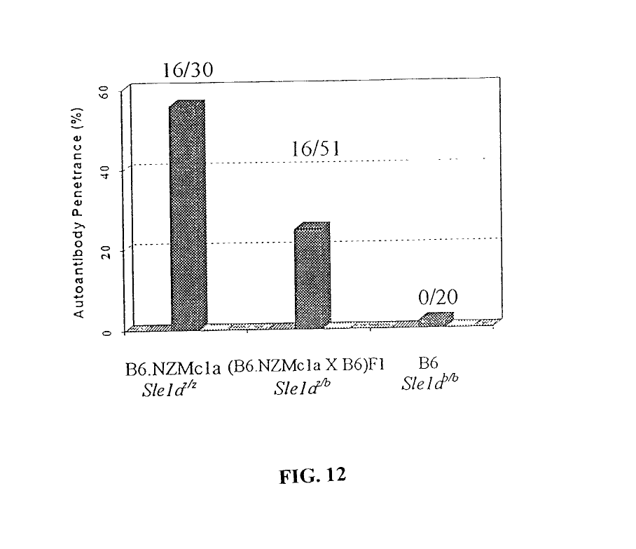

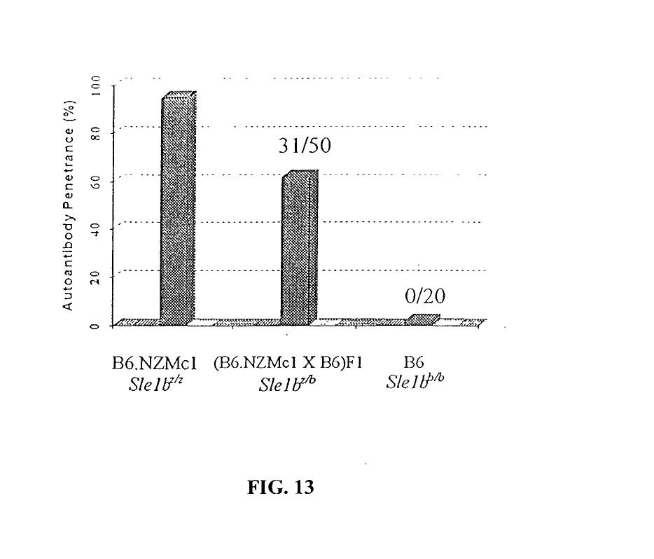

As shown in FIG. 12 and FIG. 13, the penetrance of autoantibody production is high in Sle1a^{z/z} and Sle1b^{z/z} mice, intermediate in heterozygous Sle1a^{z/b} and Sle1b^{z/b} mice, and absent in normal Sle1a^{b/b} and Sle1b^{b/b} mice. This mode of inheritance is commonly termed allele dose and indicates that the incorporation of a single Sle1a^{b} or Sle1b^{b} allele will partially suppress autoimmunity induced by the corresponding NZM2410 allele. This is consistent with the recessive inheritance of Sle1-mediated susceptibility in our original linkage analysis of autoimmune lupus nephritis in the NZM2410 mouse and suggests that transgenes carrying B6 derived alleles of these genes will at least partially suppress autoimmunity.

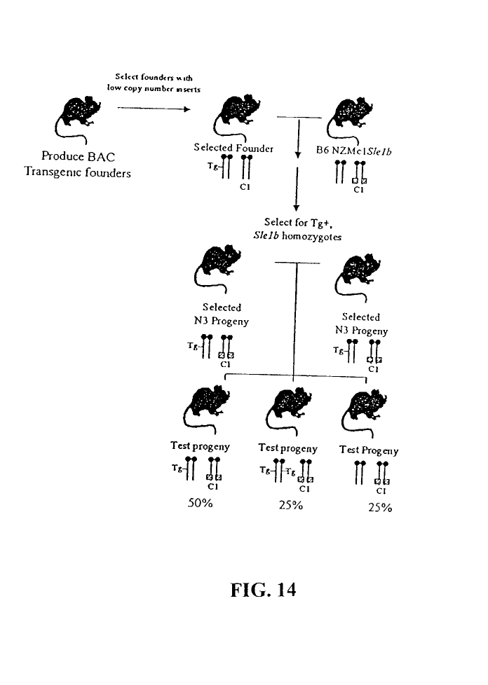

The BAC “rescue” strategy used to identify the BACs carrying Sle1a and Sle1b is diagrammed in FIG. 14. Since the strategy is identical for both intervals, the procedure will be described in the context of Sle1b. BAC transgenic mice are produced in B6 recipients by the Transgenic Core Facility at the University of Texas Southwestern Medical Center (see attached letter). The Core produces and implants a minimum of 200 injected embryos with each construct, which is usually sufficient to produce from 3 to 6 transgenic founders. The transgenic inserts in these founders generally vary in copy number and location. However, the level of expression of genes in transgenic BAC inserts increases with insert copy number, indicating that founders with single or low copy numbers of transgenic inserts will express their genes at normal physiologic levels. Founders are initially screened for transgene insert copy number using probes specific for BAC vector sequences. As diagramed in FIG. 14, low copy number B6-BAC transgenic founders are bred to B6.NZMc1Sle1b mice and B6.NZMc1Sle1 homozygotes carrying the transgene are selected (2 generations of selective breeding). These mice are intercrossed to produce a cohort of testcross littermates that carry 0, 1, or 2 copies of the inserted transgene. Statistical power estimates of the data in FIG. 13 indicate that 20 animals in each genotypic class is sufficient to achieve statistically significant suppression of autoimmunity if the BAC transgene carries a functional allele of Sle1b^{b.}

The BAC transgenic mice are assayed for autoantibody production bi-monthly from 4 through 12 months of age using procedures. At necropsy, their splenic T and B cells are tested for activation markers. Selection of BACs to be used for transgenic production was based on data obtained from the ongoing molecular genetic analysis of this region.

Data interpretation and alternative strategies. The use of the BAC rescue strategy has the advantage of allowing an in vivo functional assay to identify the physical location of a gene within a BAC contig. This is an essential element in the strategy to identify these genes, since all of their disease-relevant phenotypes are clearly only detectable in vivo. This strategy has been dramatically successful in other systems and it is reasonable to predict that it will work equally well in our lupus mice.

### EXAMPLE 4

An initial analysis of a means of identifying the gene or combination of genes within the Sle1 interval was carried out by measuring lupus in F1 hybrids between B6.NZMc1 sub-congenic intervals and NZW. This strategy was developed based on recent demonstration that (B6.NZMc1×NZW)F1 hybrids developed fatal lupus while (B6×NZW)F1 hybrids were healthy (Morel et al., 1999b). The genetic basis for the development of fatal lupus in (B6.NZMc1×NZW)F1 hybrids is complex, resulting from an epistatic interaction of homozygous Sle1 with Sle3 and Sle6 and the simultaneous inactivation of four suppressive modifiers. The key aspect relevant to an analysis of the genes within the Sle1 cluster that are required for the development of fatal disease is the dramatic variation in phenotype between (B6×NZW)F1 hybrids (healthy) and (B6.NZMc1×NZW) F1 hybrids (fatal lupus). To identify which portion(s) of the Sle1 gene cluster are required for disease development, a cohort of F1 hybrids was produced between individual sub-congenic intervals of the Sle1 cluster and NZW and aged them through 12 months to assess their susceptibility to fatal lupus nephritis.

As shown in FIG. 9, this analysis revealed several interesting features of the fashion in which this gene cluster interacts with other lupus susceptibility genes to cause severe disease. First, Sle1c is incapable of interacting with Sle3 and Sle6 to cause disease. Similarly, regions centromeric to Sle1a did not result in detectable disease. In this regard, the region centromeric to D1MIT400 in the congenic interval of B6.NZMc1 has not expressed any autoimmune component phenotypes throughout the study, thus excluding this region as a site of additional susceptibility genes. Interestingly, Sle1a and Slelb in combination (designated NZW×C1(400-206) in FIG. 9) only generate very minimal disease (4% fatal lupus). These results indicate that neither of these genes two genes in association are insufficient to regenerate the severe epistatic disease mediated by the full Sle1 interval. The strongest effect was clearly localized to a ˜9 cM interval beginning with Sle1b and extending telomeric to marker D1MIT152. This segment generated fatal lupus with a penetrance similar to that obtained for the entire Sle1 gene cluster (36% for NZW×Cl versus 39% for NZW×Cl(113-152). These results indicate that an additional susceptibility gene, which we have designated Sle1d, is located within a 9 cM interval telomeric to Sle1b. Whether Sle1d is capable of interacting with Sle3 and Sle6 to cause fatal lupus in the absence of Sle1b is an additional issue that remains to be resolved.

### EXAMPLE 5

Gene identification and candidate screening is performed utilizing a variety of techniques. Gene finding software such as GenScan, Procrustes, GeneParser and Grail is used to identify potential expressed sequences within BAC genomic sequences. These putative gene sequences are screened against a variety of EST databases (e.g., Unigene) to identify any previously cloned expressed sequences, and utilized as probes on Northern blots containing RNA isolated from a variety of different tissues. Genes within the critical region are evaluated and prioritized as candidate genes based first on expression analysis using Northern blot, RNAse protection, and RT-PCR panels from tissue obtained from both the NZM2410 and the C57BL/6J mouse strains, as previously described. Genes that show appropriate tissue expression, namely B cell expression, especially if that expression is shown to differ between the two mouse strains are evaluated to access any functional polymorphisms between the two strains.

Results with small murine gene expression microarrays (1200 genes, Clontech) demonstrate consistent variations in the gene expression patterns between normal B6 and B6.NZM congenic mice. Interestingly, B-lymphocytes from B6.Sle1 mice up-regulate the expression of a specific isoform of c-myc and other genes at two months of age, well prior to the initiation of autoantibody production.

### EXAMPLE 6

The level of microsatellite polymorphisms in human 1q21 CD2 family was evaluated in a panel of 20 Caucasians, 10 hispanics, and 10 African American, none of whom have SLE (Table 4). This analysis was designed to measure the level of polymorphism present at each of these loci. The result indicate that several of the genes are highly polymorphic and will be extremely useful for molecular typing of this region in humans. The most polymorphic sites that span the region encoding the 2B-4 Ly-9 SLAM CD48 gene cluster in humans were selected. Primers specific for Human Microsatellite Markers for the Sle1b region and the regions amplified are set forth in Table5.

TABLE 4
Microsatellite Polymorphisms in human 1q21 CD2 family
Human Polymorphisms in the CD2 Ig Family

| | CRP | CD48/SLAM | SLAM | LY-9 | 2B4 | USF-1 | SLAM |
|---|---|---|---|---|---|---|---|
| Size (bp) | 155 | 235 | 255 | 154 | 150 | 160 | 180 |
| Primer | HMS 1-18 | HMS 1-10 | HMS 1-16 | HMS 1-12 | HMS 1-19 | HMS 1-23 | HMS 1-18 |
| Repeat | CA | TG | CA | GT/GA | CA | CA | AC |
| Alleles | 6 | 4 | 2 | 10 | 6 | 5 | 3 |

Number of Alleles Detected

| | | | | | | | |
|---|---|---|---|---|---|---|---|
| Caucasian | 6 | 4 | 2 | 9 | 6 | 3 | 3 |
| Black | 5 | 3 | 2 | 7 | 2 | 5 | 3 |
| Hispanic | 3 | 3 | 2 | 4 | 2 | 2 | |

% Heterozygous.

| | | | | | | | |
|---|---|---|---|---|---|---|---|
| Caucasian | 0.89% | 0.31% | 0.74% | 0.95% | 0.50% | 0.18% | 0.69% |
| Black | 0.44% | 0.50% | 0.40% | 0.70% | 0.50% | 0.80% | 0.69% |
| Hispanic | 0.63% | 0.83% | 0.60% | 0.50% | 0.50% | 0.10% | |

TABLE 5
Human Microsatellite Markers for the Sle1b region

| | |
|---|---|
| HMS1-8 | |
| Location | Near SLAM gene |
| Forward Primer | |
| AAAAACCAAGCCACAACTGG | (SEQ ID NO:17) |
| Reverse Primer | |
| ATTTCCTCTTTGCCCTTTGG | (SEQ ID NO:18) |
| Product Length | 180 bp |
| Type of repeat | 16 AC repeats |
| Number of alleles | Three out |
| | of 29 samples |
| Product Sequence | |
| AAAAACCAAGCCACAACTGGGAGCAGATATTTA | (SEQ ID NO:19) |
| CAGTATATGTAACTGACAAAGGACTAATATCCA | |
| GAAACATGTAATGAATTCCTGTGAACACACACA | |
| CACACACACACACACACACACACAGAGAGAGAG | |
| AGAGAGGGAGACACATGAACAGCATTTCCCAAA | |
| GGGCAAAGAGGAAAT | |
| HMS1-10 | |
| Location | Between SLAM and |
| | CD48 (very close |
| | to SLAM gene) |
| Forward Primer | |
| TCCATCCTTCCAGCTCAGTC | (SEQ ID NO:20) |
| Reverse Primer | |
| TCCCATTTACCCTGTGTGATT | (SEQ ID NO:21) |
| Product Length | 235 bp |
| Type of repeat | 22 TG repeats |
| Number of alleles | Four out |
| | of 30 samples |
| Product Sequence | |
| TCCATCCTTCCAGCTCAGTCTCCTTGTTGTGTG | (SEQ ID NO:22) |
| TGTGTTTTCTCCTCTAATTTTTAACGTCTGTGG | |
| GTACATAGTAGATATACATATATGTGTGTGTGT | |
| GTGTGTGTGTGTGTGTGTGTGTGTGTGTGTGTG | |
| TATGTATATATATATGTATGGGGTATGCATATA | |
| TTTGTATGGGGTATATGAGATATGTTGATAGAG | |
| GCATGCAATATGTAATAATCACACAGGGTAAAT | |
| GGGA | |
| HMS1-12 | |
| Location | Between |
| | Ly-9 and 2b4 |
| | (closer to Ly-9) |
| Forward Primer | |
| ACACTGGAGCACCCAAATTC | (SEQ ID NO:23) |
| Reverse Primer | |
| GGAGTGCTGAAGTCCTCCAC | (SEQ ID NO:24) |
| Product Length | 154 bp |
| Type of repeat | 21 GT and |
| | 6 GA repeats |
| Number of alleles | Eleven out |
| | of 39 samples |
| Product Sequence | |
| ACACTGGAGCACCCAAATTCATAAAGCAAATAT | (SEQ ID NO:25) |
| TACCAGAGAGAGAGTGAGAGTAAGAGTGTGTGT | |
| GTGTGTGTGTGTGTGTGTGTGTGTGTGTGTGTG | |
| TGAGACAGAGAGAGAGAGACTGCAACAGAATAG | |
| TAGTGGAGGACTTCAGCACTCC | |
| HMS1-16 (aka D1S2771) | |
| Location | In SLAM gene |
| Forward Primer | |
| TCAGTTCCATAGGCTGACG | (SEQ ID NO:26) |
| Reverse Primer | |
| CATTGCTGATGCTGGAGG | (SEQ ID NO:27) |
| Product Length | 255 bp |
| Type of repeat | 12 CA repeats |
| Number of alleles | Two out of |
| | 39 samples |
| Product Sequence | |
| TCAGTTCCATAGGCTGACGAAACACAAAGTTCA | (SEQ ID NO:28) |
| GGCTACTGGCTTTGCTTCTTATCCTAGTATTAG | |
| AGTGATTTCTCCAGTGGTTCCTAGTGTCGATAT | |
| CATAAACCTTGAATGANTCAATCTGTCTCAAAC | |
| ACACACATACACACATACACACATACACACACA | |
| CACACACACACACACACTCCTGCACAGAGGGTT | |
| CTCAGTNACCATAAGTCACTCAGAGTGGAGCTG | |
| CTCCTTCCTCCAGCATCAGCAATG | |
| HMS1-18 (aka D1S2635) | |
| Location | Near C-Reactive |
| | Protein |
| Forward Primer | |
| TAGCAGATCCCCCGTC | (SEQ ID NO:29) |
| Reverse Primer | |
| TGAATCCTACCCCTAAGTAGAAT | (SEQ ID NO:30) |
| Product Length | 155 bp |
| Type of repeat | 18 CA Repeats |
| Number of alleles | Six out of |
| | 35 samples |
| Product Sequence | |
| AGCTGACATAGCAGATCCCCCGTCACCAAGTTG | (SEQ ID NO:31) |
| CGTGCACACGATGCATACACACACACACGCATG | |
| CGGCAAGCACACACACACACACACACACACACA | |
| CACACACACACTTCTCTTTCTTGCTTTGTATTA | |
| TAGATGAGATTCTACTTAGGGGTAGGATTCATT | |
| ATTCATGAAGGGTGTGGTCAGGTGAGGCATGTT | |
| GGAAGCAAAATGCGAATTAGGTAAGGTGGAGTA | |
| GAAGAGAGCT | |
| HMS1-23 | |
| Location | Near USF1 gene |
| Forward Primer | |
| ACATGAACCTGGGAGGTGAG | (SEQ ID NO:32) |
| Reverse Primer | |
| ACCATGCCTGGCTAATTTGT | (SEQ ID NO:33) |
| Product Length | 160 bp |
| Type of repeat | 18 CA repeats |
| Number of alleles | Five out of |
| | 37 samples |
| Product Sequence | |
| ACATGAACCTGGGAGGTGAGGTTGCAGTGAGTG | (SEQ ID NO:34) |
| AAAATCATGCTACTGCACTTCAGCCTGAGTGAC | |
| AGAGGGAGACTGTCTCAAAAAAAAAAAAAAAAA | |
| AACATGTGCACACACACACACACACACACACAC | |
| ACACACACACAAATTAGCCAGGCATGGT | |
| HMS1-26 (aka HMS1-25R and HMS1-4R) | |
| Location | Between Ly-9 |
| | and 2b4 |
| | (closer to 2b4) |
| Forward Primer | |
| CCCATATGCTGCTTCCAGAT | (SEQ ID NO:35) |
| Reverse Primer | |
| AAGGGTGTGTGCATGTGTGT | (SEQ ID NO:36) |
| Product Length | 255 bp |
| Type of repeat | 18 AC repeats |
| Number of alleles | Four out |
| | of 38 samples |
| Product Sequence | |
| ACACACATGCACACACCCTTTAGGATTTTATTG | (SEQ ID NO:37) |
| GAATTACATTGAATCTTGGTCGTTTACCTCTAC | |
| ACATACACACACACACACACACACACACACTGA | |
| TATCCAGTAGAGCAAGGCTTCTCACTTTGCTCT | |
| TCTTTTTTGGTTATCTTGTGCCCATACCACATT | |
| GTTCCAAATATTTTAGATTGATAAAATGTCTCC | |
| CTTCCCAAAGGTAATTACTGGCCTGACTGTGAT | |
| GGAAATCAGCACTTCCCCATATGCTGCTTCCAG | |
| AT | |

### EXAMPLE 7

Sle1

, a cluster of susceptibility genes targeting nuclear autoantigens. The Sle1 interval contains the most potent gene detected in the original genome scan, yielding a LOD score >10 from the analysis of 165 backcross progeny (Morel et al, 1994). In addition, a component phenotype analysis of B6.Sle1 demonstrated that Sle1 mediates the loss of tolerance to nuclear chromatin in both the T and B cell compartments, indicating that this gene is responsible for a key immunologic phenotype strongly associated with SLE. Further, analysis of genetic interactions among the Sle genes indicated that fatal lupus only occurred in combinations of susceptibility alleles that contained Sle1, indicating that it is essential for the development of fatal lupus in our model (Morel et al., 2000). Finally, the suppression of Sle1 by Sles1 was sufficient to fully suppress disease in this model system, again indicating that Sle1 is a key element in the initiation of systemic autoimmunity (Morel et al., 1999). These observation identify Sle1 as a key target for strategies and reagents design to suppress SLE pathogenesis.

Analysis of over 250 recombinants derived from >2000 testcross progeny localized the positions of four separate genes that contributed to the autoimmune phenotype produced by the Sle1 susceptibility interval. These genes were designated Sle1a, Sle1b, Sle1c, and Sle1d, and each has been derived on a separate sub-congenic interval. The key genes responsible for the component phenotypes contributed by Sle1 to fatal lupus in this model are Sle1a, Sle1b, and Sle1d, all of which are located within the critical interval which originally defined the location of Sle1. All three genes map within a ˜9 cM genomic region delimited by D1MIT109 and D1MIT152 on murine chromosome 1. Sle1a and Sle1b each break tolerance to chromatin, mediate significant splenomegaly, and cause a variety of activation phenotypes in B and/or T cells. Sle1d is located in a 5 cM region just telomeric to Sle1b and appears to play a key role in mediating the epistatic activation of severe disease when combined with Sle3 in a permissive genome. Sle1c is located well outside the critical interval for Sle1 near the telomere of chromosome 1 and only mediates a relatively weak breach in tolerance to a broad spectrum of nuclear antigens.

Several groups have reported associations between SLE susceptibility and regions on human chromosome 1 that are syntenic with the Sle1 cluster of murine susceptibility genes (Gibson et al., 1999; Wu et al., 1997). The initial association was observed between the Fc receptor complex at 1q21and SLE severity, predominantly in African American families (Gibson et al., 1999; Wu et al., 1997). Subsequently, Tsao and co-workers observed a strong linkage with SLE susceptibility and 1q41-1q44 via affected sib-pair analysis in a collection of families with diverse ethnic origins (Tsao et al., 1997). Currently, associations with at least one of these two regions have been detected by every group working on the genetics of SLE susceptibility in humans.

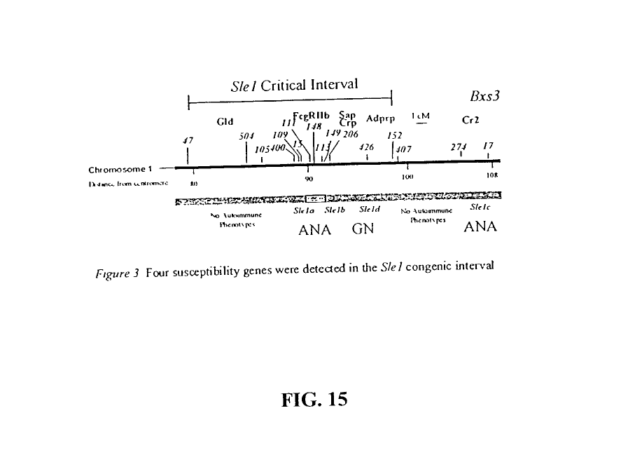

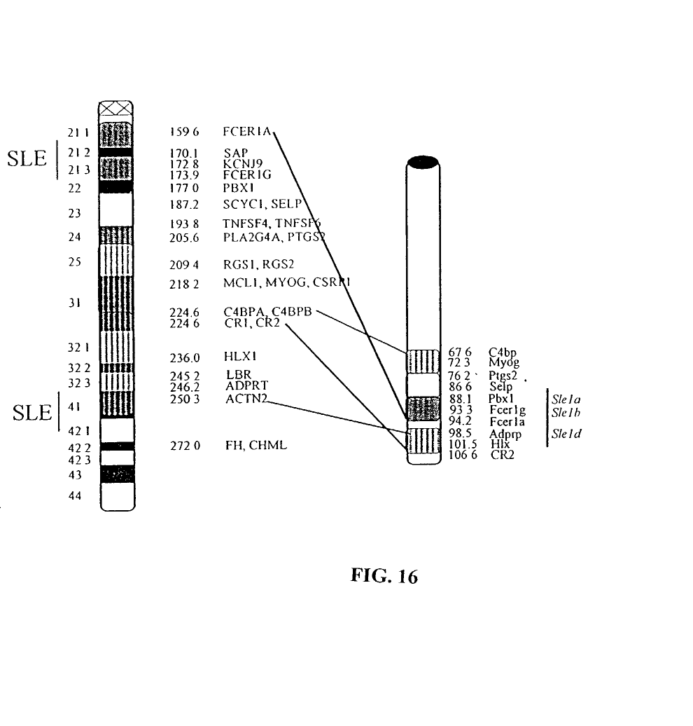

The syntenic relationship of the Sle1 gene cluster and these two SLE susceptibility intervals on human chromosome 1 is diagrammed in FIG. 16. A strong synteny is clearly observed between this region of murine chromosome 1 and these two sections of human chromosome 1q. A confusing aspect of this region is that these two segments of human 1q are in an inverted orientation on murine chromosome 1 with the breakpoint in 1q31 near CR2. As a result, genes in these two segments, which are >50 cM apart on human chromosome 1, are included within a 20 cM region of murine chromosome 1 that contains the Sle1 gene cluster. The human 1q21 region is centered on the section containing Sle1a and Sle1b. This is readily apparent based on the close proximity of these two susceptibility genes with the murine Fc receptor complex (see FIG. 15), which is in the center of human 1q21. Similarly, human 1q41 is syntenic with a large portion of the interval within Sle1 which contains Sle1d, as seen by the inclusion of murine Adprt (aka PARP) within both human 1q41 and murine chromosome 1 at 98.5 cM. Thus, both of these human genomic segments are syntenic with the single region of murine chromosome 1 that contains the Sle1 gene cluster. As a result, the identification of these murine genes is potentially highly relevant to the nature of these human SLE susceptibility genes.

Gene expression microarrays can reveal genetic pathways. The use of gene expression microarrays to cluster genes into specific pathways has been a major new technology arising from recent advances in genomics technology (reviewed in Bowtell et al., 1997). Although the efficacy of gene expression arrays is best established in the analysis of yeast gene expression, its value for the analysis of mammalian gene systems has been supported by several recent publications (Glynne et al., 1999). The use of this technology in conjunction with panels of congenic and truncated congenics facilitates the identication sets of genes with modified expression as a consequence of an SLE susceptibility gene. The potential strength of this approach is that it can be used to seek aberrant gene expression patterns in animals prior to the initiation of systemic autoimmunity. As illustrated below, this technique allows the identification of genes with disrupted expression patterns as a consequence of the intrinsic expression of a susceptibility and/or suppressive allele via the ex vivo analysis of B cells and CD4+ T cells at ages that precede the initiation of detectable humoral autoimmunity.

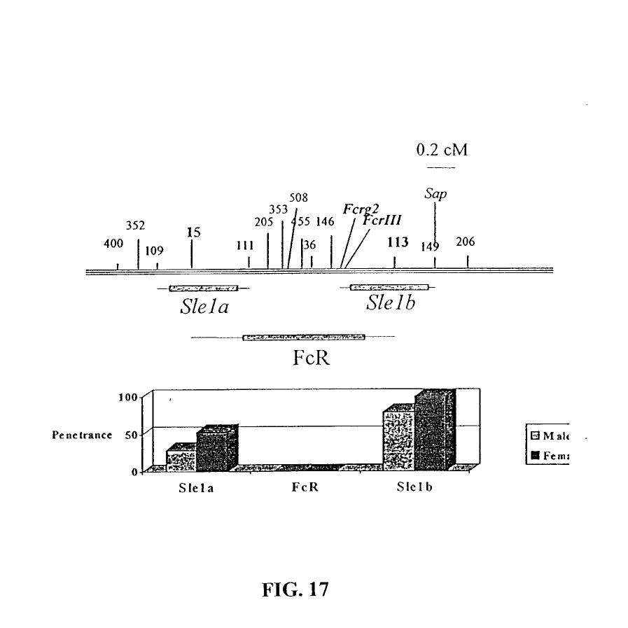

Genetic and molecular delineation of Sle1a and Sle1b. The lengths of the truncated congenic intervals that define Sle1 a and Sle1 b are presented together with their ANA phenotypes in FIG. 17. These susceptibility genes mediate highly penetrant ANA production focused on the H2AH2B-DNA subnucleosome component. The FcR congenic strain carries an interval that includes the majority of the intervening DNA between these two susceptibility genes, including the murine Fc receptor cluster. This congenic strain does not make ANA, thus excluding the Fc receptor genes as candidates for either Sle1a or Sle1b.

The termini of the critical intervals for precisely locating Sle1a and Sle1b are defined by the ends of the FcR congenic interval and the alternative ends of the congenic intervals in B6.Sle1a and B6.Sle1b. Positional cloning strategy allows the production of BAC and YAC contigs spanning each of these critical intervals.

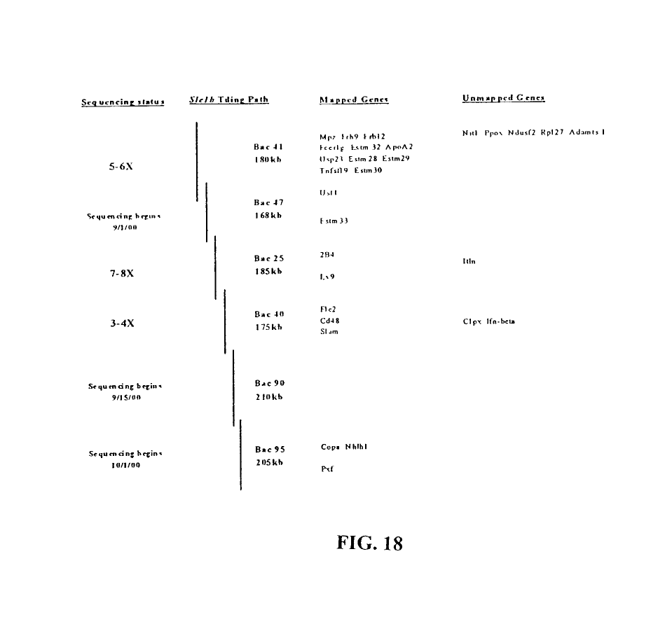

Phenotypic analysis clearly identified Sle1b as the most potent gene. The positional cloning of Sle1b required the development of a BAC contig across the Sle1b critical interval and the definition of a “minimal tiling path” of BACs required to span the entire region. Each of these BACs was then sequenced, the sequence is analyzed by a variety of methods to identify all the candidate genes, and these genes are then assessed. The analysis of Sle1b is summarized in FIG. 18.

The critical interval for Sle1b consists of approximately 800-1000 kb of genomic DNA that is spanned by six BAC clones from the deJong RPCI-26 BAC library (derived from C57BL/6). Sequencing was performed on ordered arrays of plasmid clones containing random sheared fragments prepared from each BAC in the tiling path (about 2500 reads/BAC). This sequence consisted of rough (4-5 fold coverage) to finish (8+fold coverage) quality for each of the individual BAC clones analyzed. This level of sequence quality is more than sufficient to identify all the genes within this interval via analysis with standard computer programs and strategies (the Celera sequence of the human genome is based on approximately 3-fold coverage). The completed project contains approximately 16,000 DNA sequences with an average quality read length of ˜400 bp. A total of 26 genes are identified within these segment, 19 of which are either new or previously unmapped in this region. These candidates are listed in their molecular order in FIG. 18.

Candidate gene analysis. The fundamental strategy for the identification of Sle1b is to use BAC transgenic rescue to localize it to a single BAC, and then use targeted mutagenesis in BACs and/or B6 ES cells to identify the specific gene. Previous analyses of this gene's phenotypic expression indicates that Sle1b must be expressed in B cells. In addition, the original linkage analysis indicates that Sle1 b must have functionally-relevant allelic polymorphisms between NZM2410 and C57BL/6. Based on these criteria, five strong candidate genes within the Sle1b interval were identified.

The strongest set of candidates for Sle1b are four members of the CD2 Ig supergene family located in a tight cluster (<250 kb) on BACs 25 and 40. These members are: CD48, a GPI-linked adhesion/costimulatory molecule expressed on hematopoietic cells that has been shown to potentiate T-B collaboration (Cabero et al., 1998); 2B4, an adhesion/inhibitory molecule expressed on NK and CD8 cells (also B cells in some studies) that binds with CD48 and participates in NK cell target recognition (Latchman et al., 1998); SLAM, an adhesion/costimulatory molecule that is expressed on T cells and activated B cells (Castro et al., 1999); and Ly9, an adhesion molecule of unknown function that is expressed on B cells and in the thymus. The B6 and NZM2410 alleles of CD48, Ly9, and 2B4 all differ by nucleotide changes that cause productive and non-conservative polymorphic amino acid substitutions. In addition, analysis of the expression of CD48 indicates that it is expressed at significantly decreased levels (roughly 5-fold lower) in B6.Sle1b B cells, when compared with age and sex matched B6 B cells.

This gene cluster is remarkably polymorphic, both in its content of coding sequence variations and variations in expression levels. In addition, CD48 and SLAM have been implicated as costimulatory molecules in T-B interactions and 2B4 is known to play an important role in NK cell activation/inhibition. All of these immunologic functions could impact the activation of B cells by chromatin, As a result, it is tempting to speculate that this highly polymorphic gene cluster may play a role in the regulation of B cell tolerance and that polymorphisms in their functional properties lead to variations in susceptibility to systemic autoimmunity. In this regard, the notion that a cluster of linked genes could contain alleles that impact SLE susceptibility in humans and mice is more readily acceptable then a common polymorphism in a single gene, given that the MHC gene cluster is the only other genetic region associated with autoimmunity in both species.

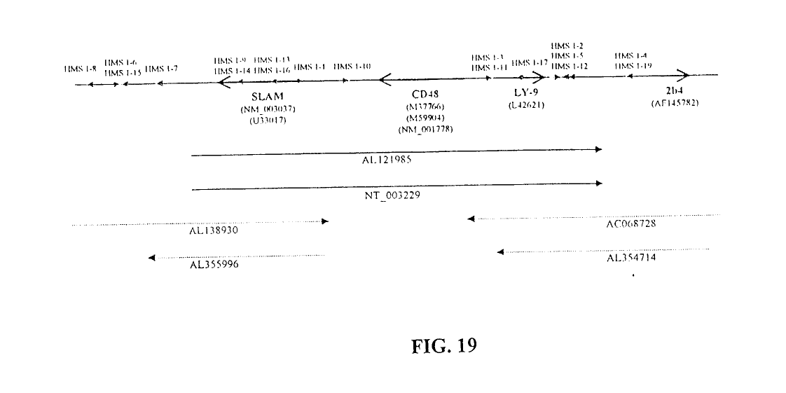

Based on the candidacy of the CD2 family as Sle1b, a search and analysis for long sequence contigs containing these genes in the public databases was performed. Based on information available in the public database, these results confirm that a homologous cluster of CD2 family genes is present within the q21 region of human chromosome 1. An analysis of these sequences revealed 18 microsatellite loci in or near the four genes in the CD2 cluster (positioned in FIG. 19). Primers were synthesized for these loci and their polymorphism assessed in a panel of 40 randomly selected human DNA samples (see Table 4). These data indicate that several of these microsatellite loci are highly polymorphic, indicating that they can be used for linkage analysis in human SLE families.

Sequence analysis of BAC41 revealed the presence of a recently described, novel member of the TNF receptor family named DEDD (Stegh et al., 1998). This gene is a ubiquitously expressed protein that contains a death effector domain similar to those of FADD and caspase-8. In addition, it has two nuclear-localization sites and a carboxy-terminal sequence that bears homology to histones. Overexpression of DEDD weakly induces apoptosis through interaction with FADD and caspase-8. Quite interestingly, Fas-ligation induces translocation of DEDD from the cytoplasm to the nucleus where it is postulated to bind DNA and shut down transcription during Fas-mediated apoptosis. Analysis reveals several sequence polymorphisms within the coding region, although none of these cause amino acid substitutions.

Gene expression microarray analysis of genetic pathways. The feasibility of using gene expression arrays to detect variations in gene expression induced by SLE susceptibility intervals was tested using CLONTECH's Mouse 1.2 array. The focus in these studies has been on seeking variations in the gene expression patterns of B and CD4+ T cells that precede the initiation of overt systemic autoimmunity. Since B6.Sle1 mice generally initiate the production of anti-chromatin antibodies beginning at 6 months of age, the gene expression patterns of 4-month old male C57BL/6 and B6.Sle1 mice were analyzed. CD4+ T and B lymphocytes were isolated from spleens of 3 mice in each strain by positive magnetic bead selection methods, and 5-10 μg total RNAs were extracted from these cells and used for microarray analysis.

Both B and T cells have significant variations in expression when B6 is compared with either B6.Sle1 or a truncated congenic containing only Sle1a and Sle1b. Most notably, c-myc and SOCS3 (suppressor of cytokines signaling protein) were significantly overexpressed in B6.Sle1 B and T cells in comparison to normal B6. However, in B6.Sle1(a+b) mice, c-myc and SOCS3 are only overexpressed in B lymphocytes. Western blotting results for c-Myc demonstrate that the upregulation of c-myc in B6.Sle1 mice results predominantly in the production of a lower molecular weight form of c-myc protein, consistent with the hypothesis that c-myc transcription is being increased via a specific activation pathway.

These results establish the feasibility of using gene expression microarrays to detect and characterize the molecular pathways that are modified by specific lupus susceptibility alleles. The data indicate that both T and B cells are activated in B6.Sle1 mice well prior to the initiation of ANA production and that this activation is being triggered via a pathway that activates the transcription of a specific isoform of c-myc. This finding correlates with those of other groups who have previously reported that c-myc overexpression correlates with the production of autoantibody (Boumpas et al., 1998). It further suggests that endogenous and exogenous factors which lead to the expression of autoimmunity might share the induction of proto-oncogene expression as a common pathogenic step with Sle1. Since c-myc plays a vital role in cell-cycle progression, deregulated expression of c-myc can overcome cell-cycle arrest and promote cellular proliferation. In the case of CD4+ T and B lymphocytes, which are frequently renewed, overexpression of c-myc could cause the dysregulation of their proliferation and, consequently, the survival of autoreactive CD4+ T and B cells.

CD4+ T cells in B6.Sle1 mice exhibit activated c-myc transcription, a phenotype that is absent in mice that only carry Sle1a and Sle1b (B6.Sle1a+b). This result, together with the observation that B cells have upregulated c-myc in both congenics, illustrates the manner in which “molecular phenotypes” can be mapped into specific intervals (<0.5 cM) using a series of congenic recombinants. This strategy will allow the identification of genes that mediate significant changes in the gene expression patterns of specific cells in the immune system, an approach which should be of general value in the analysis of genes enhancing and suppressing autoimmunity.

Experimental Design. The strategy used to identify these genes, which is illustrated by data for Sle1b, involves six basic steps. (1) fine mapping robust component phenotypes relevant to disease pathogenesis into small intervals (<0.5 cM) via the production and analysis of truncated congenic recombinants (this will remain a requisite first step in positional cloning for all four of these susceptibility genes); (2) DNA sequencing of the minimum congenic interval is determined and all of the genes residing within the interval are identified (currently, this is the most labor intensive element of positional cloning;. the completion of the genomic sequence of the mouse will allow this step to be performed on the computer); (3) assessing candidacy of all the genes within the interval via analysis of tissue distribution and functional genetic polymorphisms (this strategy involves a thorough analysis of the expression of the individual genes in the progenitor strains and a comparison of their coding sequences); (4) characterizing polymorphisms within the human homologues of any identified candidates genes and using them to assess their relevance to SLE susceptibility in humans; (5) producing a collection of BAC transgenic mice carrying each of the BACs within the tiling path and using them to localize the position of the susceptibility gene to a single BAC via a BAC transgenic rescue strategy (BAC transgenic “rescue” has become a standard approach in mouse genetics to place a gene only detectable as an in vivo phenotype into a molecular map (Probst et al., 1998)); (6) if necessary, utilizing targeted gene disruption either in ES cells or BACs to definitively identify the specific genetic polymorphism responsible for susceptibility (this step will only be necessary if an obvious candidate gene does not emerge).

BAC transgenic rescue and targeted disruptions of candidate genes in Sle1b. BAC transgenic mice with each of the six BACs in tiling path for Sle1b are specifically envisioned. These strains are produced via injection of the BACs directly into B6.Sle1b mice. The basic strategy for their analysis is illustrated in FIG. 15. ANA production in B6.Sle1 mice is inherited in an “allele dose” fashion, in which the penetrance of ANA production is significantly decreased in (B6×B6.Sle1 )F1 hybrids in comparison to B6.Sle1 homozygotes (for Sle1b, penetrance drops from 89% to 47% in heterozygotes). As a result, if the B6 homologue of Sle1b is present on the BAC carried by a particular BAC transgenic, then the ANA phenotype will be suppressed when this transgene is bred into a B6.Sle1 homozygotes.

Strategies for Sle1a, Sle1d, and Sles1. The basic approach to the identification of these genes is identical to that for Sle1b. Each of these genes are at different stages of development. Analysis of Sle1a, Sle1d, and Sles1 is useful in conjunction with BAC transgenic mice from the Sle1a interval to identify Sle1a and genes identified within the Celera database to initiate candidate gene analysis in Sle1a.

### EXAMPLE 7

The inventors sequenced the entire 940 kb Sle1b region and used a genefinding program, GenescanII and Panorama, to identify the new candidate genes. From this, the inventors have developed an extensive list of genes, a number of which are included in the present invention as prognostic of SLE, and as providing suitable therapeutic targets and reagents. These are listed (with the corresponding database accession numbers, incorporated by reference) in Table 6.

TABLE 6*

| | | Accession |
|---|---|---|
| BAC | Gene | Number |
| 41 | Usp23 - ubiquitin specific protease 23 | NM_013919 |
| | Estm29 - Ras-related GTP-binding protein | AA062310 |
| | Tnfsf19 - TNF ligand superfamily, member 19 | NM_011615 |
| | Nit1 - nitrilase1 homolog | NM_012049 |
| | Ppox - protoporphyrinogen oxidase | NM_008911 |
| | Rpl27 - ribosomal protein L27 | AF214527 |
| | Estm26 - beta-1,4-galactosyl transferase | NM_020579 |
| | Y17677 - unknown mRNA | Y17677 |
| 47 | Usf1 - upstream transcription factor 1 | NM_009480 |
| | Jam -junctional adhesion molecule | U89915 |
| 25 | 2B4 - naturaJ killer cell receptor | L19057 |
| | Ly9 - lymphocyte antigen 9 | NM_008534 |
| | ltln - intelectin | NM_010584 |
| | mCs1 - mouse homolog of human CS1 | |
| 40 | CD48 - CD48 antigen | X53526 |
| | Slam - signaling lymphocytic activation | NM_013730 |
| | molecule | |
| | Clpx - caseinolytic protease X | NM_011802 |
| 90 | Golga4 - Golgi autoantigen, golgin subfamily a | NM_018748 |
| | Tbr2 - member of T-box family | AB032373 |
| | Ly108 - lymphocyte antigen 108 | AF248635 |
| | CD84 - CD84 antigen | NM_013489 |
| 95 | Copa - coatomer alpha subunit | AJ010391 |
| | Nhlh1 - nescient helix loop helix 1 | NM_010916 |
| | Pxf - peroxismal farnesylated protein | Y09046 |
| | Nicastrin | NM_021607 |
| | Casq1 - calsequestrin | NM_009813 |
| | Atp1a4 - Na,K-ATPase alpha-4 subunit | AF164348 |
| | Pea15 - astrocytic phosphoprotein | NM_008556 |
| | Tim23 - inner mitochondrial membrane | AB021122 |
| | translocase | |
| | Human | |
| | 2b4 | AF145782 |
| | LY-9 | L42621 |
| | CS1 (19a24) | NM_021181 |
| | CD48 | NM_001778 |
| | SLAM | NM_003037 |
| | CD84 | NM_003874 |

*The bold face genes listed are expressed in B cells and, hence, are true SLE candidate genes.

F. References

The following references, to the extent that they provide exemplary procedural or other details supplementary to those set forth herein, are specifically incorporated herein by reference.

U.S. Pat. No. 4,430,434

U.S. Pat. No. 4,559,302

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,683,194

U.S. Pat. No. 4,683,195

U.S. Pat. No. 4,683,202

U.S. Pat. No. 4,727,028

U.S. Pat. No. 4,797,368

U.S. Pat. No. 4,960,704

U.S. Pat. No. 5,139,941

U.S. Pat. No. 5,168,053

U.S. Pat. No. 5,354,855

U.S. Pat. No. 5,578,832

U.S. Pat. No. 5,580,729

U.S. Pat. No. 5,614,396

U.S. Pat. No. 5,624,824

U.S. Pat. No. 5,624,830

U.S. Pat. No. 5,625,047

U.S. Pat. No. 5,837,832

U.S. Pat. No. 5,837,860

U.S. Pat. No. 5,861,242

Alt et al., J Biol. Chem., 253:1357, 1978.

Baichwal and Sugden, In: Gene transfer, Kucherlapati (ed.), NY Plenum Press, 117-148, 1986.

Berzal-Herranz et al., Genes and Devel., 6:129-134, 1992.

Bowtell, Nature Genetics, 21:25-32, 1999.

Castro et al., J. Immunol. 163:5860-5870, 1999.

Cech et al., Cell, 27:487-496, 1981.

Chalfie et al., Science, 263:802-805, 1994.

Chang et al., Hepatology, 14:134A, 1991.

Chen and Okayama, Mol. Cell. Biol. 7:2745-2752, 1987.

Chowrira et al., J. Biol. Chem., 269:25856-25864, 1994.

Chowrira et al., Biochemistry, 32:1088-1095, 1993.

Clark et al., Human Gene Ther., 6:1329-1341, 1995.

Clynes and Ravetch, Immun. 3:21, 1995.

Clynes et al., Science 279:1052-1054, 1998.

Coffin, In: Virology, Fields et al. (eds.), New York: Raven Press, pp. 1437-1500, 1990.

Cohen and Eisenberg, Annu.Rev.Immunol. 9:243-269, 1991.

Cohen and Eisenberg, Annu.Rev.lmmunol. 9:243-269, 1991.

Colberre-Garapin et al., J. Mol. Biol., 150:1, 1981.

Cotten et al., Proc. Nat'l Acad. Sci. USA, 89:6094-6098, 1992.

Couch et al., Am. Rev. Resp. Dis., 88:394-403, 1963.

Coupar et al., Gene, 68:1-10, 1988.

Curiel, In: Viruses in Human Gene Therapy, Vos (Ed.), Carolina Academic Press, Durham, N.C., pp 179-212, 1994

Eddleston et al., Genomics 56:149-159, 1999.

EPO 0273085

Fechheimer et al., Proc. Nat'l Acad. Sci. USA 84:8463-8467, 1987.

Flotte et al., Gene Ther., 2:29-37, 1995.

Fodor et al., Nature, 364(6437):555-6, 1995.

Forster and Symons, Cell, 49:211-220, 1987.

Friedmann, “Progress toward human gene therapy,” Science, 244:1275-1281, 1989.

Gaffney et al., Proc. Nat'l Acad. Sci. USA 95:14875-14879, 1998.

Gaffney et al., Proc. Nat'l Acad. Sci. USA 95:14875-14879, 1998.

Gerlach et al., Nature 328:802-805, 1987.

Ghosh and Bachhawat, In: Liver Diseases, Targeted Diagnosis and Therapy Using Specific Receptors and Ligands, Wu and Wu (Eds.), Marcel Dekker, New York, pp 87-104, 1991.

Ghosh-Choudhury et al., EMBO J., 6:1733-1739, 1987.

Gibson et al., In Lupus: Molecular and Cellular Pathogenesis. Kammer and Tsokos, editors. Human Press, Inc, Totowa. 557-573, 1999.

Glynne et al., Curr. Opin. Immunol., 12:210-214, 2000.

Gomez-Foix et al., J. Biol. Chem., 267:25129-25134, 1992.

Gopal, Mol. Cell. Biol., 5:1188-1190, 1985.

Graham and Prevec, Biotechnology, 20:363-390, 1992.

Graham and Van Der Eb, Virology, 52:456-467, 1973.

Graham et al., J. Gen. Virol., 36:59-72, 1977.

Grunhaus and Horwitz, Seminar in Virology, 3:237-252, 1992.

Grunhaus and Horwitz, Seminar in Virology, 3:237-252, 1992.

Hacia et al., Nature Genet., 14:441-449, 1996.

Harley et al., Curr. Op. Immun. 10:690-696, 1998.

Harley et al., Curr. Op. Immun., 10:690-696, 1998.

Haseloff and Gerlach, Nature, 334:585-591, 1988.

Hennonat and Muzyczka, Proc. Nat'l. Acad. Sci. USA, 81:6466-6470, 1984.

Hersdorffer et al., DNA Cell Biol., 9:713-723, 1990.

Herz and Gerard, “Proc. Nat'l. Acad. Sci. USA 90:2812-2816, 1993.

Holmstrom et al., Anal. Biochem. 209:278-283, 1993.

Horwich et al. J. Virol., 64:642-650, 1990.

Izui et al., Int. Rev. Immunol. 11:211-230, 1994.

Jones and Shenk, Cell, 13:181-188, 1978.

Joyce, Nature, 338:217-244, 1989.

Kanedaetal., Science, 243:375-378, 1989.

Kaplitt et al., Nature Genetics, 8:148-154, 1994.

Karlsson et al., EMBO J., 5:2377-2385, 1986.

Kasahara et al., Science, 266:1373-1376, 1994.

Kato et al., J. Biol. Chem., 266:3361-3364, 1991.

Kaufmnan, Methods in Enzymology, 185:537-566, 1990.

Kelleher and Vos, Biotechniques, 17(6):1110-1117, 1994.

Kim and Cech, Proc. Nat'l Acad. Sci. USA, 84:8788-8792, 1987.

Klein et al., Nature, 327:70-73, 1987.

LaFace et al., Virology, 162:483-486, 1988.

Latchman and Reiser, Eur. J. Immunol. 28:4325-4331, 1998.

Latchman et al., J. Immunol., 161:5809-5812, 1998.

Laughlin et al., J. Virol., 60:515-524, 1986.

Le Gal La Salle et al., Science, 259:988-990, 1993.

Lebkowski et al., Mol. Cell. Biol., 8:3988-3996, 1988.

Levrero et al., Gene, 101:195-202, 1991.

Lieber and Strauss, Mol. Cell. Biol., 15: 540-551, 1995.

Luo et al., Blood,82 (Supp.): 1,303A, 1994.

Markowitz et al., J. Virol., 62:1120-1124, 1988.

McCarty et al., J. Virol., 65:2936-2945, 1991.

McLaughlin et al., J. Virol., 62:1963-1973, 1988.

McPherson, Genome Research 7:1111-1113, 1997.

Michel and Westhof, J. Mol. Biol., 216:585-610, 1990.

Miller, Curr. Top. Microbiol. Immunol., 158:1, 1992.

Mohan et al., J. Clin. Invest. 101:1362-1372, 1998.

Mohan et al., J. Clin. Invest. 103:1685-1695, 1999.

Mohan et al., J. Immunol., 159:454-465, 1997.

Mohan et al., J. Immunol., 159:454-465, 1997.

Mohan et al., J. Immunol., 162:6492-6502, 1999.

Morel et al., J. Immunol., 158:6019-6028, 1997.

Morel et al., Immun., 11:131-139, 1999.

Morel et al., Immun. 1(3):219-229, 1994.

Morel et al., Mamm. Genome, 10:176-181, 1999.

Morel et al., Proc. Nat'l Acad. Sci. USA 97:6670-6675, 2000.

Moser et al., Proc. Nat'l Acad. Sci. USA, 95:14869-14874, 1998.

Muzyczka, Curr. Top. Microbiol. Immunol., 158:97-129, 1992.

Newton et al., Nucl. Acids Res. 21:1155-1162, 1993.

Nicolas and Rubinstein, In: Vectors: A survey of molecular cloning vectors and their uses, Rodriguez and Denhardt (eds.), Stoneham: Butterworth, pp. 494-513, 1988.

Nicolau and Sene, Biochem. Biophys. Acta, 721:185-190, 1982.

Nicolau et al., Methods Enzymol., 149:157-176, 1987

Ohi et al., Gene, 89L:279-282, 1990.

Osoegawa et al., Genomics, 52:1-8, 1998.

Palukaitis et al., Virology, 99:145-151, 1979.

Paskind et al., Virology, 67:242-248, 1975.

Pease et al., Proc. Nat'l Acad. Sci. USA, 91:5022-5026, 1994.

Perales et al, Proc. Nat'l Acad. Sci. USA, 91:4086-4090, 1994.

Perriman et al., Gene, 113:157-163,1992.

Potter et al., Proc. Nat'l Acad. Sci. USA, 81:7161-7165, 1984.

Probst et al., Science, 280:1447-1451, 1998.

Prody et al., Science, 231:1577-1580, 1986.

Racher et al, Biotechnology Techniques, 9:169-174, 1995.

Ragot et al., Nature, 361:647-650, 1993.

Rasmussen et al., Anal. Biochem, 198:138-142, 1991.

Ravetch et al., Annu. Rev. Immunol., 16:421-432, 1998.

Reinhold-Hurek and Shub, Nature, 357:173-176, 1992.

Remington's Pharmaceutical Sciences, 15th Edition, Chapter 61, pages 1035-1038 and 1570-1580.

Renan, Radiother. Oncol., 19:197-218,1990.

Rich et al., Hum. Gene Ther., 4:461-476, 1993.

Ridgeway, In: Vectors: A survey of molecular cloning vectors and their uses. Rodriguez and Denhardt, eds. Stoneham: Butterworth, pp. 467-492, 1988.

Rippe et al., Mol. Cell Biol., 10:689-695, 1990.

Rosenfeld et al., Science, 252:431-434, 1991.

Rosenfeld et al., Cell, 68:143-155, 1992.

Roux et al., Proc. Nat'I Acad. Sci. USA, 86:9079-9083, 1989.

Running et al., BioTechniques, 8:276-277, 1990.

Salmon etal., J. Clin. Invest. 97:1348-1354, 1996.

Samulski et al., EMBO J., 10:3941-3950, 1991.

Samulski et al., J. Virol., 63:3822-3828, 1989.

Santerre et al., Gene, 30:147, 1984.

Sarver et al., Science, 247:1222-1225, 1990.

Scanlon et al, Proc. Nat'l Acad. Sci. USA, 88:10591-10595, 1991.

Shai et al., Human Molecular Genetics, 8:639-644, 1999.

Shelling and Smith, Gene Therapy, 1:165-169, 1994.

Shoemaker et al., Nature Genetics, 14:450-456, 1996.

Sioud et al., J. Mol. Biol., 223:831-835, 1992.

Sobel et al., J. Immunol. 162:2415-2421, 1999.

Stegh et al., EMBO J. 17:5974-5986, 1998.

Stratford-Perricaudet and Perricaudet., In: Human Gene Transfer, Eds, Cohen-Haguenauer and Boiron, Editions John Libbey Eurotext, France, pp. 51-61, 1991.

Stratford-Perricaudet et al., Hum. Gene. Ther., 1:241-256, 1991.

Symons, Annu. Rev. Biochem., 61:641-671, 1992.

Symons, Nucl. Acids Res., 9:6527-6537, 1981.

Temin, In: Gene Transfer, Kucherlapati (ed.), New York: Plenum Press, pp. 149-188, 1986.

Thompson et al., Nature Genet., 9:444-450, 1995.

Top et al., J. Infect. Dis., 124:155-160, 1971.

Tratschin et al., Mol. Cell. Biol., 4:2072-2081, 1984.

Tratschin, et al., Mol. Cell. Biol., 5:3258-3260, 1985.

Tsao et al., J. Clin. Invest 103:1135-1140, 1999.

Tsao et al., J. Clin. Invest. 99:725-731, 1997.

Tur-Kaspa et al., Mol. Cell Biol., 6:716-718, 1986.

Underhill et al, Genomics, 55:185-193, 1999.

Vollrath et al, Genome Research 9:150-157, 1999.

Wagner et al., Science, 260:1510-1513, 1990.

Wakeland et al., J. Clin. Immunol., 17:272-281, 1997.

Walsh et al., J. Clin. Invest., 94:1440-1448, 1994.

Wandstrat et al Am. J. Hum. Genet. 62:925-936, 1998.

Wei, et al., Gene Therapy, 1:261-268, 1994.

Winchester, In Systemic lupus erythematosus. R.G.Lahita, editor. Churchill Livingstone, New York. 65-85, 1992.

Wong et al., Gene, 10:87-94, 1980.

Wu and Wu, Adv. Drug Delivery Rev., 12:159-167, 1993.

Wu and Wu, J. Biol Chem., 262:4429-4432, 1987.

Wu et al., Arthritis Rheum, 40:S316, 1997.

Wu et al., J. Clin. Invest. 100:1059-1070, 1997.

Yang et al., Proc. Nat'l Acad. Sci. USA, 87:9568-9572, 1990.

Yoder et al., Blood, 82 (Supp.): 1:347A, 1994.

Yuan and Altman, Science, 263:1269-1273, 1994.

Yuan et al., Proc. Nat'l Acad. Sci. USA, 89:8006-8010, 1992.

Zhou et al., Exp. Hematol. (NY), 21:928-933, 1993.

Zhou et al., J. Exp. Med., 179:1867-1875, 1994.

What is claimed is:

1. A method of screening for susceptibility to a systemic autoimmune disorder comprising, screening for at least one mutation within the SLE-1B loci.

2. The method of claim 1, wherein said systemic autoimmune disorder is systemic lupus erythematosus.

3. The method of claim 1, wherein said method of screening comprises FISH.

4. The method of claim 1, wherein said method of screening comprises the use of a DNA array.

5. The method of claim 4, wherein said DNA array comprises a DNA chip.

6. The method of claim 1, wherein said method of screening comprises PCR amplification.

7. The method of claim 1, wherein said method of screening comprises hybridizing a polynucleotide probe.

8. The method of claim 7, wherein said method of screening comprises Southern blotting.

9. The method of claim 1, wherein said at least one mutation occurs in a gene or genes within the SLE-1B loci.

10. The method of claim 8, wherein said gene encodes SLAM.

11. The method of claim 8, wherein said gene encodes Ly-9.

12. The method of claim 8, wherein said gene encodes 2B4.

13. The method of claim 8, wherein said gene encodes CD48.

14. The method of claim 8, wherein said gene encodes CD84.

15. The method of claim 8, wherein said gene encodes LY108.

16. The method of claim 8, wherein said gene encodes CS 1.

17. The method of claim 8, wherein said gene encodes DEDD.

18. The method of claim 8, wherein said gene encodes NIT1.

19. The method of claim 8, wherein said gene encodes USF1.

20. The method of claim 8, wherein said gene encodes GOLGA4.

21. A method of treating a systemic autoimmune disorder comprising administering to a host a construct comprising wild-type sequence from within the SLE-1B loci.

22. The method of claim, wherein said autoimmune disorder is systemic lupus erythematosus.

23. The method of claim 21, wherein said wild-type sequence comprises sequence encodes CD48, CD84, SLAM, 2B4, LY108, CS1, DEDD, NIT1, LY9, USF1 or GOLGA4.

24. A method of screening for susceptibility of a host to systemic lupus erythematosus comprising:
a) obtaining a tissue sample from said host comprising nucleic acid;
b) isolating nucleic acid from said sample; and
c) assaying said sample for the presence of at least one mutation within the SLE-1B loci;
wherein said at least one mutation is indicative of an increased susceptibility to developing systemic lupus erythematosus.

25. The method of claim 24, wherein said host is a human.

26. The method of claim 24, wherein said assaying comprises FISH.

27. The method of claim 24, wherein said assaying comprises the use of a DNA array.

28. The method of claim 27, wherein said DNA array comprises a DNA chip.

29. The method of claim 24, wherein said assaying comprises PCR amplification.

30. The method of claim 24, wherein said assaying comprises hybridizing a polynucleotide probe to said nucleic acid.

31. The method of claim 30, wherein said assaying comprises Southern Blotting.

32. A DNA array comprising polynucleotide molecules complementary to genetic sequence within the SLE-1B loci, wherein said polynucleotide molecules comprise mutant sequence indicative of a systemic autoimmune phenotype.

33. A DNA array for use in the detection of genetic susceptibility to systemic lupus erythematosus comprising nucleic acid probes, said probes comprising sequence complementary to one or more mutated regions of genes selected from the group consisting of Cd48, Cd84, Slam, 2B4, Ly108, Cs1, Dedd, Nit1, Ly9, Usf1 and Golga4.

34. A method of screening for therapeutics for treating systemic autoimmunity comprising a mouse with at least one mutation within the SLE1-B loci, wherein said mouse develops an autoimmune phenotype.

35. The method of claim 34, further characterized as administering to said mouse a compound and monitoring the progression of said autoimmune phenotype in said mouse.

36. The method of claim 34, wherein said autoimmune disorder is systemic lupus erythematosus.

37. The method of claim 34, wherein said at least one mutation occurs in a gene or genes within the SLE-1B loci.

38. The method of claim 30, wherein at least one mutation occurs within the sequence encoding CD48, CD84, SLAM, 2B4, LY108, CS1, DEDD, NIT1, LY9, USF1 or GOLGA4.
